(19) United States
(12) Patent Application Publication
(10) Pub. No.: US 2024/0206774 A1
(43) Pub. Date: Jun. 27, 2024

(54) DETERMINATION OF THE PRESENCE OF A TARGET SPECIES

(71) Applicant: ADAPTIVE DIAGNOSTICS LIMITED, Nottingham (GB)

(72) Inventors: Mustafa KHRAISHI, Nottingham, Nottinghamshire (GB); Fergus MORRIS, Nottingham, Nottinghamshire (GB)

(21) Appl. No.: 18/558,168

(22) PCT Filed: Apr. 29, 2022
(86) PCT No.: PCT/EP2022/061606

§ 371 Date: Oct. 30, 2023

(30) Foreign Application Priority Data
Apr. 29, 2021 (GB) 2106143.7

(51) Int. Cl. A61B 5/1455; A61B 5/00; G06T 7/00; G06T 7/90; G16H 30/00

(52) U.S. Cl. CPC A61B 5/1455; A61B 5/4842; A61B 5/7267; G06T 7/0012; G06T 7/90; G16H 30/00

(57) ABSTRACT

A computer-implemented method of determining the presence of a target species in a fluid sample comprises: receiving a captured image including an image capture portion of a sample vessel, the image capture portion exposing a fluid sample contained within the sample vessel, wherein an optical property of the fluid sample is indicative of the presence of the target species; processing the captured image to generate optical property data representative of the optical property of the fluid sample; and determining whether the target species is present in the fluid sample based on the optical property data. A sample vessel is also provided.

The present invention relates to methods, including computer-implemented methods for determining the presence of a target species in a fluid sample. The present invention also relates to a sample vessel which may be used according to methods of the invention, and for a system for determining the presence of a target species in a fluid sample.

### BACKGROUND TO THE INVENTION

It is well-known that the presence or absence of a disease may be evaluated by testing fluid samples such as blood samples and saliva samples from a subject. However, these processes often require a user to attend a dedicated clinic in order for a sample to be taken, and then complicated lab-based tests to be performed in order to determine the subject's disease status. It is therefore desirable to provide methods and systems which may be used by a subject straightforwardly, and at home, in order to perform the same tests. The present invention aims to address this issue.

### SUMMARY OF THE INVENTION

Broadly speaking, the present invention is directed towards systems and methods for determining a status or progression of a disease, based on an optical property of a fluid sample which is obtained from a subject. The present invention is advantageous because it allows a user to straightforwardly test themselves for a disease using simple equipment, and eliminates the need for them to travel to e.g. a hospital to be tested. Accordingly, a first aspect of the invention provides a computer-implemented method of determining the presence of a target species in a fluid sample obtained from a subject, the method including the steps of: receiving a captured image including an image capture portion of a sample vessel, the image capture portion exposing a fluid sample contained within the sample vessel, wherein an optical property of the fluid sample is indicative of the presence of the target species; processing the captured image to generate optical property data representative of the optical property of the flu id sample; and determining whether the target species is present in the fluid sample based on the optical property data.

The term “target species” here is referred to any substance or entity which may be present in the fluid sample. For example, the target species could cover a target molecule, a target particle, a target compound, a target complex, a target micro-organism (such as a bacteria, virus, or any other pathogen, or an antigen associated with the micro-organism in question), a target analyte or the like. The presence of the target species is preferably indicative of the presence of a disease (and accordingly, the absence of the target species is preferably indicative of the absence of the disease). Similarly, an amount or concentration of the target species in the fluid sample may be indicative of the presence of a disease, or the progression of a disease. Throughout this application, the term “disease status” is used to cover, for example, the presence/absence of a disease, the progression of a disease, or any other property/parameter associated with the disease, e.g. the type of disease. The progression of a disease may be measurable on a clinical scale. Alternatively put, the presence, expression or activity of the target species may be associated with the disease, or the upregulated expression or activity of the target species may be associated with the disease. The mechanism by which the presence of the target species gives rise to a change in the optical property of the fluid sample is discussed in more detail later.

The captured image preferably includes an identification code, such as a quick response (QR) code which encodes additional data about the test. The additional data may include one or more of: an identity of the subject, a date/time at which the test is taken, a unique ID of the test session, information about the disease, information about the first reagent, information about the second reagent, a test version, and the like. Herein, a “version” of the test is indicative of a specific configuration of the test, such as the reagents used, and the specific type of sample vessel. This is because the optical properties of the test will vary depending on these specific properties, and reliable results can only be obtained by comparing like-for-like versions). For example, if in one implementation, the target species gives rise to a colour change from transparent to orange, this could not be meaningfully compared to a test in which the colour change is from transparent to black. Similar considerations apply if the sample vessels are made from different materials. This is why it is important to store information about the exact specification used for a given test.

We now discuss the features of the captured image in more detail. The sample vessel preferably includes an image capture region, which is a part of the vessel in which the fluid sample is visible, so that an image of it can be captured. Preferably, the identification code is located in the image capture region, so that the captured image includes a fluid sample region (i.e. a region in which the fluid sample is visible), and an identification code region (i.e. a region in which the identification code is visible).

In some cases, the image capture region of the sample vessel may be defined by the identification code. For example, an outside boundary or periphery of the image capture region may be defined by an outer boundary of the identification code. When the image capture region of the sample vessel is defined by the identification code, the identification code preferably includes a cut-out region, or a transparent region, the fluid sample being visible through that region. The fluid sample region of the captured image in such cases is defined by the boundaries of the transparent region or cut-out region. Throughout the application, the transparent region or cut out region may be referred to as a “window”. In some cases, the window may be surrounded on all sides by the identification code, but alternatively, the window may be located at an edge or corner of the identification code. When the image capture region is defined by the identification code in this manner, it is possible to minimize the amount of surface area taken up by the identification code on the sample vessel, meaning that smaller sample vessels can be used, which in turn means that less material is required, and smaller fluid sample volumes can be used.

Processing the captured image preferably comprises identifying the image capture region in the captured image. This may be done by identifying defining features in the captured image. For example, the identification code may include boundary features which indicate the location of the edge of the identification code, and identifying the image capture region may comprise detecting the boundary features in the captured image. The boundary features may then correspond to the edge of the image capture region. The remainder of the captured image may then be discarded. This is effective when the identification code includes a window, as discussed above, because it means that it is necessary only to identify one boundary, which encompasses the identification code, and the fluid sample region.

As discussed, the image capture region includes the fluid sample region, i.e. the region of the image where the fluid sample is visible. Accordingly, processing the captured image comprises identifying the fluid sample region within the image capture region. As above, the image capture region, or the identification code may include boundary features indicating the boundary of the fluid sample region.

The computer-implemented method preferably includes extracting the image data pertaining to the fluid sample region from the captured image. The step of processing the captured image may comprise processing the image data from the fluid sample region of the captured image. The computer-implemented method may also include extracting the additional data from the identification code region of the captured image. The extracted image data from the fluid sample region of the captured image may then be stored in association with the additional data extracted from the identification code region of the captured image. Once these data have been extracted, and optionally stored in association with each other, the captured image may be discarded, in order to ensure that information which is not useful is not stored in a given memory location.

We now discuss the optical property in more detail. Broadly speaking, the optical property of the fluid sample which is used to provide information about the presence of the target species is the colour of the fluid sample. Accordingly, the step of processing the captured image comprises determining the colour of the fluid sample in the fluid sample region of the captured image. This may include extracting colour data from the image data corresponding to the fluid sample region of the captured image. Herein, “colour data” should be considered to cover data representative of one or more of: hue, saturation, shade, luminosity, brightness, fluorescence, phosphorescence, colourfulness, chroma, chrominance, luma, U-value, and V-value. It will be appreciated that other parameters representative of the colour of the fluid sample may also be taken into account.

The image capture region, preferably a portion of the identification code may include one or more colour calibration features. These may be the same features as the boundary features, and as such may be used for both purposes. The colour calibration features are preferably standard examples of colours which can be used to determine accurately, the colour of the fluid sample.

The final step of the computer-implemented method of the first aspect of the invention is determining whether the target species is present in the fluid sample, based on the optical property data. Determining whether the target species is present in the fluid sample may further include determining an amount of target species in the fluid sample, either in the form of a concentration or an absolute measurement. Other “amount” measurements are also envisaged.

In some cases, determining whether the target species is present in the fluid sample comprises applying an analytical model to the optical property data, the output of the model being a value indicative of the presence and/or amount of the target species in the fluid sample. The analytical model preferably receives an input, and generates an output. The input may either be the raw optical property data, and may include the calibration features. Alternatively, the method may include a further step of adjusting the optical property data based on the calibration features present in the captured image, and the input may be the adjusted optical property data. As previously discussed, the output may be in the form of a simple “yes/no” answer regarding the presence of the target species, or a numerical value indicating an amount of the target species. In alternative cases, the computer-implemented method may further include a step of determining a clinical output from the results of the previous determination step. For example, the clinical output may be information regarding the status or progression of a disease, i.e. an indication of whether or not the test subject from whom the sample was received has a particular condition, or an indication of the stage of a condition. The optical property of the fluid sample may also be used to identify a disease which the subject is suffering from, e.g. different colours may correlate with different conditions.

In preferred cases, the analytical model is a machine-learning model, such as a regression model or a classification model, particularly a binary classification model (which may be able to output e.g. a “positive” or “negative” result for the presence of a disease, in one preferred case). It is preferred to use non-adjusted data (see previous paragraph) as the input for the machine-learning model, since a trained machine-learning model is likely to be able to cope with the presence of the calibration features. In some cases, determining whether the target species is present in the fluid sample may comprise: applying a first analytical model to the raw optical property data to generate a first output, and separately applying a second analytical model to the adjusted optical property data to generate a second output, wherein the first analytical model is a machine-learning model, and the second analytical model is a non-machine-learning model. The computer-implemented method may further comprise: determining a first confidence score for the first output, and determining a second confidence score for the second output, determining which of the first confidence score and the second confidence score is greater, and selecting the corresponding output as the value indicative of the presence of the target species. The computer-implemented method may comprise comparing the first confidence score and the second confidence score to a threshold (or optionally, to respective first and second thresholds), and if the confidence score falls below the threshold (or the respective first or second threshold), determining that the test is inconclusive.

A second aspect of the invention provides a computer-implemented method for constructing the machine-learning model for determination of the presence of a target species in a fluid sample, the method including the steps of, for each of a plurality of samples: capturing an image including an image capture portion of a sample vessel, the image capture portion exposing a fluid sample contained within the sample vessel, wherein an optical property of the fluid sample is indicative of the status or progression of the disease; processing the captured image to generate optical property data representative of the optical property of the fluid sample; and assigning a value representative of the status or progress of the disease based on the optical property data; and storing the respective associations between the optical property data and the values representative of the status or progression of the disease; using the stored associations to train the machine-learning model. In some cases, captured image, the optical property data, and the assigned value may be received in a web application, and stored in a memory. The machine-learning model may be trained using the Amazon Web Services command line interface (AWS CLI), and the AWS Management Console, using the AWS SageMaker service. Preferably, training the machine-learning model will comprise using a supervised learning algorithm, such as the XGBoost algorithm¹, or Linear Learner algorithm². 1 https://docs.aws.amazon.com/sagemaker/latest/dg/xgboost.html2 https://docs.aws.amazon.com/sagemaker/latest/dg/linear-learner.html

It should be explicitly understood that the machine-learning model that may be used in the determining step of the first aspect of the present invention may be generated or constructed using the computer-implemented method of the second aspect of the invention.

A third aspect of the present invention provides a computer program comprising instructions which, when the program is executed by a program, cause the computer to carry out the computer-implemented method of the first aspect of the invention. A fourth aspect of the present invention provides a computer-readable data carrier having stored thereon the computer program of the third aspect of the invention. A fifth aspect of the present invention provides a computer program comprising instructions which, when the program is executed by a program, cause the computer to carry out the computer-implemented method of the second aspect of the invention. A sixth aspect of the present invention provides a computer-readable data carrier having stored thereon the computer program of the fifth aspect of the invention.

A seventh aspect of the invention provides a method of determining the presence of a target species in a fluid sample, the method including: capturing an image including an image capture portion of a sample vessel, the image capture portion exposing a fluid sample contained within the sample vessel, wherein an optical property of the fluid sample is indicative of the presence of the target species; and performing the method of the first aspect of the invention. It should be noted that the optional features which have been set out above with respect to the first aspect of the invention may also be incorporated into the seventh aspect of the invention.

In order to ensure that the captured image is sufficiently high quality to obtain a meaningful result from the method of the seventh aspect of the invention, the method may further include steps which guide the user to position an image capture device in an ideal location/orientation, or which automatically ensure that the image is captured at the appropriate time. For example, capturing the image may include: detecting by an image capture device the locations of the boundary features in the field of view (FOV) of the image capture device; determining a deviation of the locations of the boundary features from an optimal arrangement of the locations of the boundary features in the FOV of the image capture device; and if the determined deviation is greater than a predetermined threshold deviation, providing, via a display of the image capture device, instructions to the user of the image capture device to adjust the position of the image capture device relative to the sample vessel.

Alternatively, or additionally, capturing the image may comprise: displaying to a user of an image capture device, via a display of the image capture device, one or more alignment features to be aligned with the boundary features of the identification code in the FOV of the image capture device; detecting when the alignment features are within a predetermined threshold distance from the respective boundary features in the FOV of the image capture device; and in response to detecting that the alignment features are within the predetermined threshold distance from the respective boundary features in the FOV of the image capture device, automatically capturing the image.

Similar steps may also be taken, in which capturing the image further comprises: detecting by an image capture device a brightness parameter of the FOV of the image capture device; determining a deviation of the brightness parameter from an optimal brightness parameter value or range of values; and if the determined deviation is greater than a predetermined threshold deviation, providing, via a display of the image capture device, instructions to the user of the image capture device to capture the image in a different location which is either brighter or less bright, determining on the direction of the deviation. Here, the brightness parameter may be, for example, the luminosity or the luminance of the FOV of the image capture device. Preferably, a statistical parameter which is representative of the brightness is used, such as a mode, mean, or median luminosity or luminance value. Preferably, this user guidance feature is performed in real-time such that the user continuously receives feedback until the FOV has the desired properties (i.e. in terms of position, orientation and brightness). When the desired properties are obtained, the method may include a step of automatically capturing the image.

Thus far, we have described features pertaining to image capturing, image processing, and the subsequent processing of the image data. However, before all of this is possible, it is necessary first to obtain a sample from a subject, and then to process it to ensure that the desired optical property can be measured. Broadly speaking, in order to obtain a meaningful clinical output from a fluid sample, it is necessary to cause the target species within the sample to interact in some way with a reagent, thereby giving rise to the measurable change in the optical property of the fluid sample. In some cases, a single interaction between the target species in the user's bodily fluid may be sufficient. Alternatively, it may be necessary first to cause the bodily fluid to interact with a first reagent, for example to prepare the target species, and then with a second analyte. Discussed in detail later, in order to achieve this, a dedicated sample vessel may be used which enables a test subject, or clinical practitioner to perform the two reactions with ease.

In some cases, the method may further include: obtaining the fluid sample in the sample vessel, which comprises the steps of: providing the sample vessel to a subject, the sample vessel containing at least a first reagent; obtaining a sample of a bodily fluid from the subject in the sample vessel, the bodily fluid containing the target species to be tested; mixing at least the first reagent with the bodily fluid in the sample vessel, to provide the fluid sample, wherein the optical property of the fluid sample depends on a degree of interaction between the first reagent and the target species.

In the two-reagent cases, the method may further include: obtaining the fluid sample in the sample vessel, which comprises the steps of: providing the sample vessel to a subject, the sample vessel containing at least a first reagent and a second reagent; obtaining a sample of a bodily fluid from the subject in the sample vessel, the bodily fluid containing the target species to be tested; mixing at least the first reagent with the bodily fluid in the sample vessel, wherein interaction between the first reagent and the target species generates an intermediate reagent, mixing the intermediate reagent with the second reagent, to generate the fluid sample wherein the optical property of the fluid sample depends on a degree of interaction between the intermediate reagent and the second reagent. Examples of first and second reagents which may be used in implementations of the present invention are set out in detail in GB 2018532.8 filed on 26 Nov. 2020, the contents of which are incorporated herein by reference.

For completeness, we now describe a mechanism by which the change in optical property may be effected.

In some cases, the target species may react directly with the first reagent, the reaction causing the change in optical property, such as a colour change. Alternatively, the target species may interact with a first component of the first reagent to generate an intermediary reagent, which itself interacts with a second component of the first reagent, which causes the change in optical property.

In preferred examples, however, there is a two-step process employing a first reagent and a second reagent. This is particularly important in scenarios in which some time is required for the target species in the bodily fluid requires time to interact with the first reagent, to generate the intermediate reagent. For example, the target species may interact with the first reagent, the interaction generating an intermediate reagent. This interaction may take some time, during which it may be undesirable for the intermediate reagent to react with the second reagent. The intermediate component may interact with the second reagent, or a component thereof, in order to cause the change in optical property. A detailed description of the nature of the reagents which may be used in this invention is set out at the end of the detailed description.

Examples of intermediate reagent/optical property-changing component pairs include:
An acidic/basic component and a pH indicator (e.g. methyl violet, malachite green, thymol blue, methyl yellow, bromophenol blue, congo red, methyl orange, screened methyl orange, bromocresol green, methyl red, methyl purple, litmus, bromocresol purple, bromothymol blue, phenol red, neutral red, naphtholphthalein, cresol red, cresolphthalein, phenolphthalein, thymolphthalein, alizarine yellow R, indigo carmine). Universal indicator may also be used.
Glucose and glucose oxidase.
Starch and iodine.
Horseradish peroxidase and an appropriate substrate, and horseradish peroxidase-like DNAzymes.

In these cases, the second reagent preferably includes a colour-changing component and a liposome encapsulating a chemical entity which interacts with the colour-changing component of the second reagent to give rise to a change in an optical properly in the form of a colour change. As used herein, a ‘liposome’ refers to a vesicle comprising a membrane formed by a lipid bilayer, surrounding an aqueous cavity. In preferred cases, liposomes according to the present disclosure comprise a membrane formed by a phospholipid bilayer.

It will be appreciated that in order to release the chemical entity from the liposome, the liposomal membrane will need to be broken down somehow. To that end, the first reagent preferably includes a component which, in the presence of the target molecule, generates, forms, or liberates a liposomal membrane-destabilizing agent. Examples of useful liposomal membrane-destabilizing agents are discussed in detail at the end of the description of this application.

As mentioned previously, the present invention may employ a sample vessel which is adapted to aid the user in performing the method of the seventh aspect of the invention. Broadly speaking, an eighth aspect of the invention provides a sample vessel which facilitates the interaction of the bodily fluid, and a first reagent. Certain implementations of the eighth aspect of the invention facilitate the interaction of the bodily fluid with the first reagent, and then separately, the interaction of the intermediate reagent with the second reagent, thereby providing an integrated piece of apparatus for obtaining the required fluid sample. Specifically, an eighth aspect of the present invention provides a sample vessel for collection of a bodily fluid from a subject, and for preparing a fluid sample, the vessel comprising: a sample collection assembly, including a sample collection element which is configured to receive and retain a sample of a bodily fluid from a subject; and a containment component including: an outer housing; an inner mechanism located inside the outer housing, the inner mechanism having an inner surface defining a cavity arranged to receive the sample collection element of the sample collection assembly; and a first reagent chamber; wherein the sample collection assembly is positionable in the cavity in a testing position in which the sample collection element is in fluid communication with the first reagent chamber. Herein, when two regions are in “fluid communication” with each other, it should be understood to mean that a fluid, preferably a liquid, is able to flow unimpeded from one to the other, in other words the two regions are not isolated from each other by some kind of impermeable component.

When in the testing position, the sample collection element is preferably configured to release the bodily fluid into the first reagent chamber. For example, the sample collection element may include some kind of absorbent body, such as a sponge, and when the sample collection element is in the testing position, the absorbent body may be in a compressed state in which it releases the absorbed bodily fluid into the first reagent chamber. The sample collection assembly may include a handle portion at a proximal end, and the sample collection element at an opposing, distal end. The sample collection assembly may be generally elongate, having a longitudinal axis defining an axial direction, wherein the proximal end is axially opposite to the distal end. When the sample collection element comprises an absorbent body, the sample may be retrieved by a user sucking and chewing on the absorbent body so that it absorbs the user's saliva. To that end, the absorbent body may be coated in a substance such as a flavouring or citric acid which promotes salivation. In alternative cases, the bodily fluid may include a sample of tissue/organ of a subject, and/or of a product produced by a tissue/organ of a subject. The sample may form part or all of the reaction medium for the assay. A product produced by a tissue/organ of a subject may e.g. be a product of secretion (e.g. a glandular secretion, milk, colostrum, tears, saliva, sweat, cerumen, mucus), sputum, semen, vaginal/cervical fluid, blood (plasma, serum), cerebrospinal fluid (CSF), a product of excretion, faeces, or urine, skin or hair. In preferred cases, the sample includes a generally colourless, transparent fluid in order emphasize the colour change.

The containment component may also be generally elongate, and may share its longitudinal axis with the sample collection assembly. The sample collection assembly may be movable, preferably in the axial direction, relative to the containment component. This enables the sample collection assembly to be positioned in the testing position. While the sample collection assembly is in the testing position, it is desirable that the target species in the bodily fluid be able to interact with the first reagent, which may take some time. Accordingly, it is desirable for the sample collection assembly and the inner mechanism of the containment component to be fixed relative to each other when the sample collection assembly is in the testing position. The sample vessel may therefore preferably include a locking mechanism configured to limit or prevent relative axial movement between the sample collection assembly and the inner mechanism when the sample collection assembly is in the testing position. An implementation of the locking mechanism is described in detail later in this application with reference to the drawings. Example locking mechanisms may include a locking component on the sample collection assembly (preferably an end or outer surface thereof), and a complementary locking component on the inner mechanism (preferably an end or inner surface thereof, e.g. the internal cavity wall). For example, the locking mechanism may include a slot and a tab, a recess and a protrusion, interlocking lugs, or equivalent. The locking mechanism may be a frictional locking mechanism. When the sample collection assembly and the inner mechanism are fixed relative to each other, there may no relative movement between them.

In a first relative configuration of the inner mechanism and the outer housing of the containment component, the first reagent chamber may be defined by a first chamber-defining surface of the outer housing and a first chamber-defining surface of the inner mechanism. Here, a chamber-defining surface is any surface which defines at least a portion of the first reagent chamber. In some cases, the first chamber-defining surface of the inner mechanism and the first chamber-defining surface of the outer housing may define an entire inner surface of the first reagent chamber.

In addition to the first reagent chamber, the containment may comprise a second reagent chamber. In the first relative configuration between the inner mechanism and the outer housing, the second reagent chamber is preferably isolated from the first reagent chamber by a barrier portion of the inner mechanism or a barrier portion of the outer housing. Herein “barrier portion” is used to refer to any element or portion of a component which prevents fluid communication between the first reagent chamber and the second reagent chamber. In the first relative configuration between the inner mechanism and the outer housing, the second reagent chamber is preferably defined by a second chamber-defining surface of the outer housing, and a second chamber-defining surface of the inner mechanism. In some cases, the second chamber-defining surface of the inner mechanism and the second chamber-defining surface of the outer housing may define an entire inner surface of the second reagent chamber.

The preceding paragraphs refer to a “first relative configuration between the inner mechanism and the outer housing”. This is because it is preferable that the inner mechanism is movable relative to the outer housing, between the first relative configuration and a second relative configuration. Preferably, the two are movable relative to each other in the axial direction, as previously defined. For completeness, we note that when the sample collection assembly and the inner mechanism are fixed relative to each other, the locked-together assembly is movable in full, relative to the outer housing. Throughout the application, we will only refer to the relative motion of the inner mechanism, though.

Preferably, the inner mechanism is only movable relative to the outer housing (i.e. between the first and second configurations) under the application of a force of sufficient magnitude, and in the correct direction. Specifically, in the first relative configuration between the inner mechanism and the outer housing, at least a portion of an outer surface of the inner mechanism is in contact with at least a portion of an inner surface of the outer housing, such that the contact between the two limits the relative axial movement between the inner mechanism and the outer housing, in the absence of an axial force of sufficient magnitude. Specifically, the contact may give rise to a frictional force between the outer surface of the inner mechanism and the inner surface of the outer housing.

When the inner mechanism and the outer housing are in the second relative configuration, the first reagent chamber and the second reagent chamber are in fluid communication with each other. When the sample vessel is configured in this manner, a user may insert the sample collection assembly into the cavity defined by the inner mechanism, i.e. into the testing position, where the fluid contained in the sample collection assembly can be combined with a first reagent in the first reagent chamber. Then, once the bodily fluid has been given sufficient time to react with the first reagent, the user can move the inner mechanism (and locked sample collection assembly) relative to the outer mechanism, into the second relative configuration. At this point, because the first reagent chamber and the second reagent chamber are now in fluid communication, the mixture of the bodily fluid and the first reagent can then mix with a second reagent which was previously stored in the second reagent chamber. In this way, the device facilitates a two-stage reaction process, which is entirely self-contained. The inner mechanism may include the barrier portion, and the relative movement between the inner mechanism and the outer housing to the second relative configuration may cause the barrier portion to move away from a position in which it isolates the first reagent chamber from the second reagent chamber.

In some cases, the sample vessel may comprise a porous membrane. Specifically, when the inner mechanism and the outer housing are in the second relative configuration, the porous membrane may separate the first reagent chamber and the second reagent chamber. The porous membrane preferably does not prevent fluid communication between the first reagent chamber and the second reagent chamber. In some cases, the sample fluid which is collected may include DNA molecules or fragments of DNA molecules. It may be desirable, in some implementations of the invention, to prevent the DNA molecules from passing from the first reagent chamber to the second reagent chamber, or vice versa. Accordingly, the average pore size of the membrane is approximately 3 kilodalton, which is sufficient to prevent DNA molecules from passing through the membrane, but which allows passage of other biomolecules. More broadly speaking, the pore size may be no less than 0.1 kDa, 0.5 kDa, 1.0 kDa, 1.5 kDA, 2.0 kDA, or 2.5 kDa. The pore size may be no more than 3.0 kDA, 3.5 kDa, 4.0 kDa, 4.5 kDa, 5.0 kDA, 5.5 kDa, 6.0 kDa or 10 kDa. Commercially available membranes exist which have the required properties.³ It has been shown that the use of such membranes achieves the required technical effects.⁴ 3 See, for example https://shop.pall.com/us/en/products/centrifugal-filters/nanosep4 Elsaadani Moez, Durand Noel, Sorli Brice, Guibert Benjamin, Atelier Pascaline, Montet Didier, “Aptamer assisted ultrafiltration cleanup with high performance liquid chromatography-fluorescence detector for the determination of OTA in green coffee,” Food Chemistry, 310 (2010) https://doi.org/10.1016/j.foodchem.2019.125851.

In some cases, in order to ensure the passage of sample fluid through the porous membrane from the first reagent chamber to the second chamber (or vice versa), the first reagent chamber may be at a higher pressure than the second reagent chamber (or vice versa).

We now discuss some additional structural details of the sample vessel. In some implementations, a base of the outer housing may include: a base surface and a protrusion, the protrusion having a protrusion outer surface; a base of the inner mechanism may include a base surface axially opposite the base surface, and a recess having an recess inner surface; and in the first relative configuration between the inner mechanism and the outer housing: the first reagent chamber may be defined by at least the base surface of the inner mechanism and the opposing base surface of the outer housing; and at least a portion of the protrusion may be located inside the recess, such that the second reagent chamber is defined by the recess inner surface and a portion of the protrusion outer surface. In the first relative configuration between the inner mechanism and the outer housing, at least a portion of the surface of the protrusion may be in contact with a rim of the recess, or with the recess inner surface. In addition, the protrusion outer surface may include a lower portion which is proximal to the base surface, and an upper portion which is distal to the base surface; and in the first relative configuration between the inner mechanism and the outer housing: the first reagent chamber may be defined by the base surface of the inner mechanism, the base surface of the outer housing and the lower portion of the protrusion outer surface; and the second reagent chamber may be defined by the recess inner surface and the upper portion of the protrusion outer surface. Furthermore, the base of the inner mechanism may include a wall defining the recess, an inner surface of the wall being the recess inner surface, and further comprising a wall outer surface; and in the first relative configuration between the inner mechanism and the outer housing, the first reagent chamber may be defined by at least the base surface of the outer housing, the axially opposing base surface of the inner mechanism, the wall outer surface and the lower portion of the protrusion outer surface. In the second relative configuration between the inner mechanism and the outer housing, a gap may be defined between the protrusion outer surface and the rim of the recess or the recess inner surface, such that the first reagent chamber and the second reagent chamber are in fluid communication at least via that gap.

In order to maintain the inner mechanism and the outer housing in the second relative configuration, the containment component may further comprise a second locking mechanism, configured to prevent relative axial movement between the inner mechanism and the outer housing (when they are in the second relative configuration). The second locking mechanism may take the same form as the first locking mechanism, discussed earlier in this application. When the second locking mechanism is engaged, the sample collection assembly, the inner mechanism and the outer housing are preferably all fixed in position relative to each other.

The first reagent chamber may contain a first reagent, which may be a solid reagent or a liquid reagent. The second reagent chamber may contain a second reagent, which may also be a solid reagent or a liquid reagent.

The outer housing preferably includes a peripheral wall, and in the first relative configuration between the inner mechanism and the outer housing, the first reagent chamber is preferably defined at least by an inner surface of the peripheral wall. It is the peripheral wall of the outer housing of the sample vessel which preferably includes the image capture region. The image capture region is preferably transparent or translucent.

When the sample vessel defines only a first reagent chamber, which is in fluid communication with the sample collection element when in the testing position, it is preferable that the first reagent chamber is visible through the image capture region. In those arrangements in which there are both a first reagent chamber and a second reagent chamber, it is preferable that in the second relative configuration between the inner mechanism and the outer housing, the single chamber formed by fluid communication between the first reagent chamber and the second reagent chamber is visible through the image capture region. In all cases, the image capture region is preferably a flat region of the peripheral wall, for improved image capture of e.g. a square or rectangular identification code. The peripheral wall, save for the image capture region, is preferably a cylindrical or substantially cylindrical wall.

The peripheral wall preferably further comprises an identification code, which may be a quick response (QR) code. The identification code may encode additional data, which may include one or more of an identity of the subject, a date/time at which the test is taken, a unique ID of the test session, information about the disease, information about the first reagent, information about the second reagent, a test version, and the like. The identification code is preferably located in the image capture region, as defined above.

In some cases, the image capture region of the sample vessel may be defined by the identification code. For example, an outside boundary or periphery of the image capture region may be defined by an outer boundary of the identification code. When the image capture region of the sample vessel is defined by the identification code, the identification code preferably includes a cut-out region, or a transparent region, the fluid sample being visible through that region. The fluid sample region of the captured image in such cases is defined by the boundaries of the transparent region or cut-out region. In some cases, the window may be surrounded on all sides by the identification code, but alternatively, the window may be located at an edge or corner of the identification code. When the image capture region is defined by the identification code in this manner, it is possible to minimize the amount of surface area taken up by the identification code on the sample vessel, meaning that smaller sample vessels can be used, which in turn means that less material is required, and smaller fluid sample volumes can be used.

The image capture region, preferably a portion of the identification code may include one or more colour calibration features. These may be the same features as the boundary features, and as such may be used for both purposes. The colour calibration features are preferably standard examples of colours which can be used to determine accurately, the colour of the fluid sample. The colour calibration features may also include greyscale portions (e.g. a so-called “mid grey”) which may be used to calibrate the brightness of an image of the image capture region.

Previously, we have defined methods and computer-implemented methods of determining the presence of a target species, and a sample vessel which may advantageously be used to obtain the sample from a test subject. We now combine these aspects of the invention into a system which may be used to perform the various methods and computer-implemented methods of the invention.

Accordingly, a ninth aspect of the present invention provides a system for determining the presence of a target species in a fluid sample, the system comprising: an image capture device configured to capture an image including an image capture portion of a sample vessel, the image capture portion exposing a fluid sample contained within the sample vessel, wherein an optical property of the fluid sample is indicative of the status or progression of the disease; and processing means for performing the computer-implemented method of the first aspect of the invention. In some cases, the processing means comprises a single processor which is configured to receive the captured image, to process the captured image, and to determine the presence of the target species in the fluid sample, based on the optical property data. The processor may include a plurality of modules which are configured to perform the various functions. The modules may be implemented in the form of physical hardware modules, or software modules, each containing instructions in the form of code, which causes the processor to execute the operations required to implement the respective step of the computer-implemented method of the first aspect of the invention.

The image capture device is preferably located on a mobile device, such as a smart phone, a tablet, or a laptop computer. In some cases, at least part of the processing means may be located on the mobile device. For example, processing means on the mobile device may be configured to receive the captured image from the image capture device, and to generate the optical property data. The mobile device may then be configured to transmit the optical property data to a server or other processor, which is configured to perform the determination step, e.g. by applying the trained machine learning algorithm to the optical property data. Example system diagrams are provided in the next section of this application.

It will be appreciated that the sample vessel forms an integral part of the method of e.g. the seventh aspect of the invention. Accordingly, the system of the ninth aspect of the invention preferably includes the sample vessel of the eighth aspect of the invention.

In the above, the optional features which are set out with respect to a particular aspect of the invention in question should be understood to apply to any other aspect of the invention, to the extent that the feature is compatible with that other aspect of the invention. Further optional features of the invention are set out in the following sections.

### BRIEF DESCRIPTION OF THE DRAWINGS

The present invention will now be described with reference to the attached drawings, in which:

FIG. 1 shows a high-level system diagram of a system which may be used to implement the methods of the present invention.

FIG. 2 shows a flowchart of the steps of a method which may be used to determine the presence of a target species according to the present invention.

FIGS. 3A to 3E show various stages of use of a sample vessel according to the eighth aspect of the present invention.

### DETAILED DESCRIPTION OF THE DRAWINGS

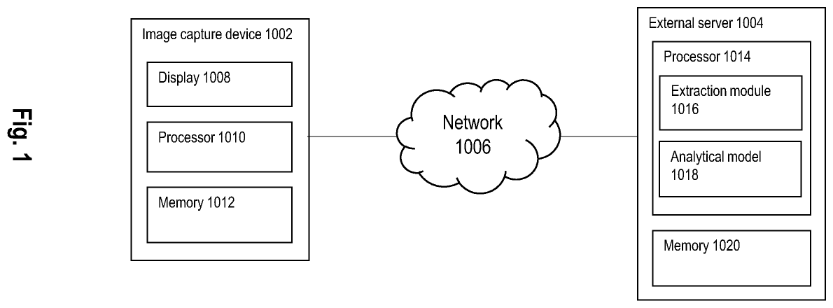

FIG. 1 is an example of a system 1000 according to e.g. the ninth aspect of the present invention. The system includes an image capture device 1002 which is connected to an external server 1004 via a network 1006, which may be a wired or wireless network. In alternative configurations, the image capture device 1002 may be connected directly to the external server, e.g. via a wired or wireless connection. The image capture device 1002 includes a display 1008, a processor 1010, and a memory 1012. The external server 1004 includes a processor 1014 which itself includes an extraction module 1016 and an analytical model 1018, as well as a memory 1020.

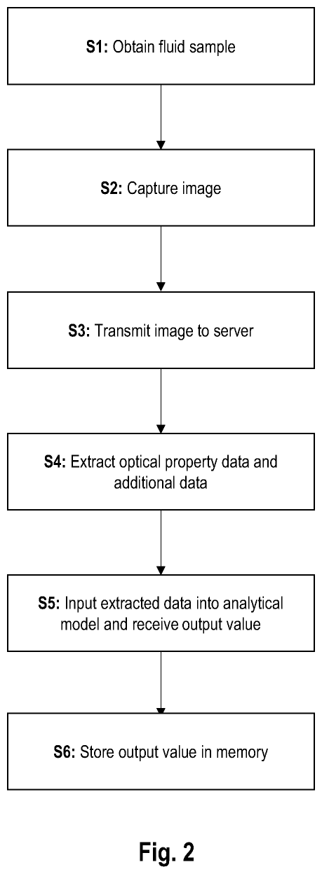

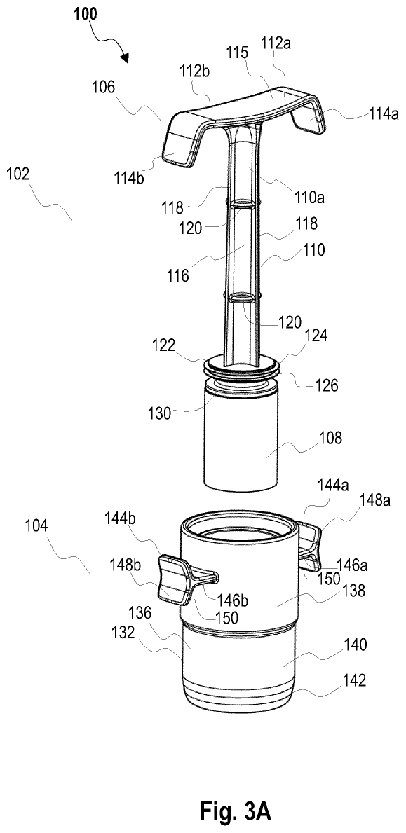

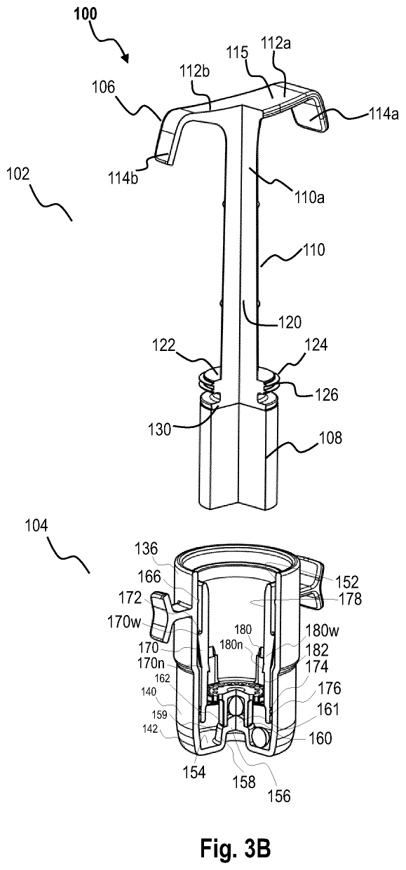

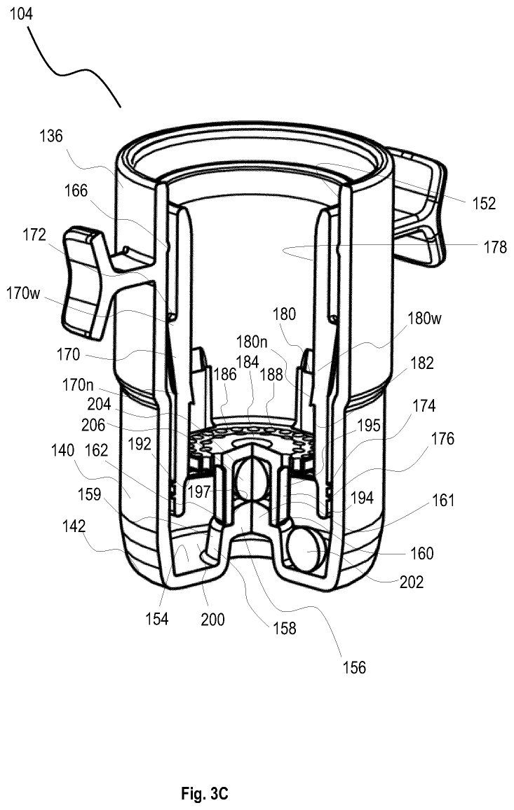

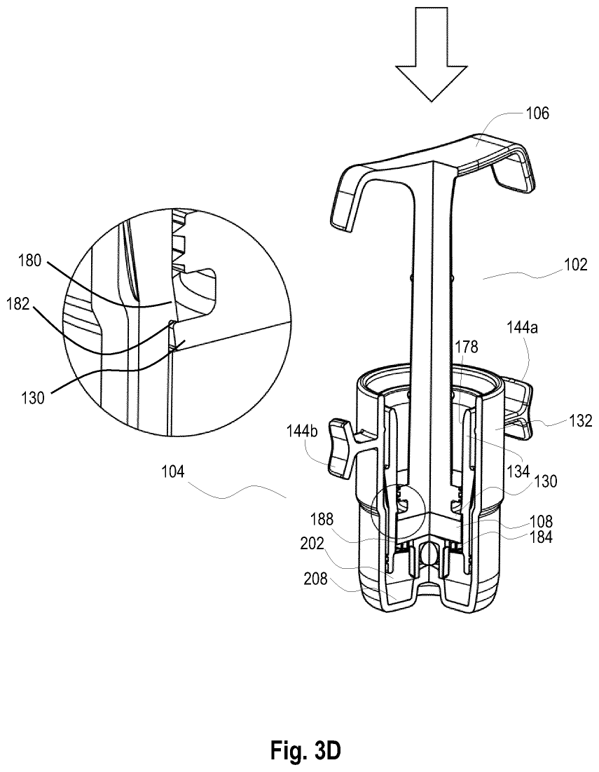

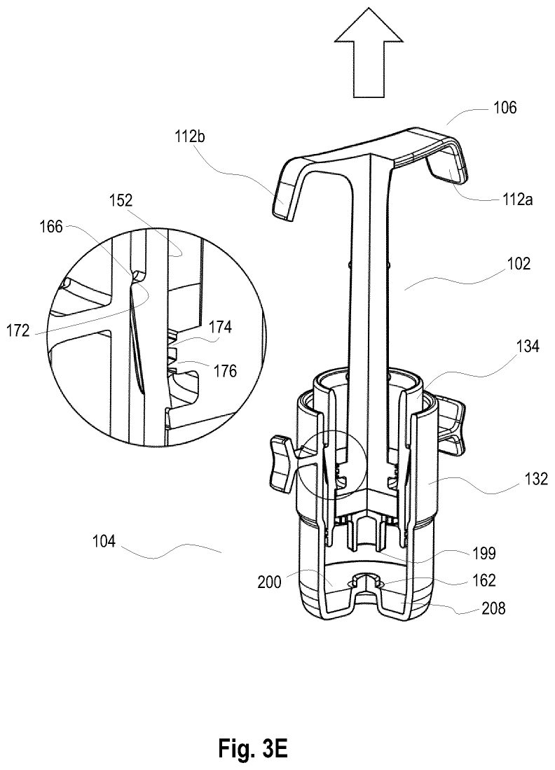

FIG. 2 is a flowchart showing the steps of a method which may be performed by the system 1000 of FIG. 1. In a first step S100, a fluid sample is obtained, using, for example the sample vessel 100 of FIGS. 3A to 3E. The process by which the fluid sample is obtained will be described in detail later on with reference to FIGS. 3A to 3E. Once the fluid sample has been obtained in step S1, an image of a fluid sample region of the sample vessel 100 is obtained using the image capture device 1002. During the image capture process of step S2, the user may receive guidance via the display 1008 of the image capture device 1002 to ensure that the image quality is sufficiently high that a meaningful output can be derived from it. This guidance may take a number of forms:
The image capture device 1002, specifically the processor 1010 thereof, may measure e.g. a brightness parameter, such as the average luminosity of the image in the field of view, and compare it to a range of acceptable values. If the image is too bright, or too dark, then the image capture device 1002 may instruct the user to capture the image in an alternative location, or to adjust the lighting in the environment in which they are taking the image.
The image capture device 1002, specifically the processor 1010 thereof, may be configured to detect boundary features of the identification code in the image capture region of the sample vessel 100, and based on their relative location in the FOV of the image capture device 1002, instruct the user to make various adjustments to the placement of the image capture device 1002 relative to the sample vessel 100:
   i. By determining the relative distances between the boundary features, or alternatively by comparing the area of the identification code in the image (i.e. the area bounded by the boundary features) with the area of the whole image, it is possible to determine whether the image capture device 1002 is too close or too far away from the sample vessel 100. The user may then be instructed to move the image capture device 1002 further from or closer to the sample vessel 100 accordingly.
   ii. By assessing the shape of the region bounded by the boundary features in the FOV of the image capture device 1002, it may be possible to determine whether the sample vessel 100, specifically the identification code in the image capture region thereof, is at too much of an angle to the image capture device 1002. For example, by comparing the apparent height and width of the identification code in the image in the FOV of the image capture device 1002 (e.g. by comparing the distances between various boundary features), it is possible to determine how the user needs to reposition the image capture device 1002 in order to ensure that the identification code is parallel to the image capture device 1002.

In step S3, the image capture device 1002 transmits the captured image to the external server 1004. In some cases, if the image is not to be sent immediately, it may be stored in the memory 1012 of the image capture device 1002. The captured image is subsequently received at the external server 1004 (this is not shown as a step in the flowchart). The captured image may include a fluid same region in which the fluid sample is visible, e.g. through a transparent portion of the sample vessel 100, and an identification code region in which the identification code is visible. In step S4, the extraction module 1016 of the processor 1014 may be configured to extract optical property data from at least the fluid sample region of the captured image, and to extract the additional data (defined previously) from the identification code region of the capture image. In preferred cases, the optical property data is colour data, and may include calibration data derived from e.g. colour calibration features of the captured image. In other cases, the processor may include an image adjustment module which is configured to adjust the colour data based on the colour calibration features to generate adjusted colour data. In step S5, the extracted optical property data is input into analytical model 1018, whereupon it is processed to output a value representative of the presence and/or amount of the target species in the fluid sample. In cases where image adjustment takes place beforehand, in step S5 the adjusted optical property data may be input into the analytical model 1018. In step S6, the output value is stored in the memory 1020 of the external server 1004 in association with the additional data extracted by the extraction module 1016 from the identification code region of the captured image. Optionally, the output value may be transmitted back to the image capture device 1002 via network 1006 for display to a user. In an alternative arrangement (not shown), the extraction module 1016 may be part of the processor 1010 of the image capture device 1002. In a further alternative arrangement (also not shown), the analytical model 1018 may also be present on the image capture device 1002, and all of the steps shown in FIG. 2 may be performed by the image capture device 1002 (except the transmitting and receiving steps, which of course would not be necessary).

We now describe the structure and usage of the sample vessel 100 which is used to obtain the fluid sample from the user. The sample vessel 100 is shown in FIGS. 3A to 3E, in various stages of use.

FIG. 3A shows sample vessel 100 which includes two main components: sample collection assembly 102 and containment component 104. Sample containment assembly 102 includes a handle 106, and an absorbent body 108 which are connected via an elongate shaft 110. Specifically a proximal end 110a of the elongate shaft 110 is integrally formed with a central point P of handle 106. Handle 106 itself includes wings 112a and 112b, which extend outwards from the central point in opposite directions generally perpendicular to the direction of a longitudinal axis of the elongate shaft 110. The ends of the wings 112a, 112b which are distal to the central point P of the handle 106 are curved downwards, to form downward curving portions 114a, 114b. The upper surface 115 of the handle 106 is slightly concave, for increased comfort as a user presses down on it with their thumb. The elongate shaft 110 is formed of a central cylindrical shaft 116, and four radially extending reinforcement members 118, which are integrally formed with the central cylindrical shaft 116, and act to prevent bending of the elongate shaft 110 when the handle 106 is pressed with the thumb. There are two circumferential reinforcing rings 120 positioned along the elongate shaft 110, and integrally formed with the central shaft 116 and the reinforcement members 118, in order to provide additional structural strength. Distal end of the shaft 110b includes a wider cylindrical portion 122, which includes two circumferential tapered lips 124, 126. Spaced from the wider cylindrical portion 122 by an intervening portion 128 is a base plate 130, which forms the attachment portion for the absorbent body 108. The base plate 130 is in the form of a thin, circular disc integrally formed with the elongate shaft 110, and having a plane which is generally perpendicular to the longitudinal axis A. The radial extent of the base plate 130 and the tapered lips 124, 126 is approximately the same. The absorbent body 108 is cylindrical and parallel to the elongate shaft 110. As discussed previously, the absorbent body 108 may be in the form of a sponge which is coated in a substance such as citric acid, or a flavouring, which simulates production of saliva by a user.

The containment component 104 is generally cylindrical, having a longitudinal axis which is parallel to the longitudinal axis A. The containment component 104 includes an outer housing 132 and an inner mechanism 134 (not visible in FIG. 3A, described in detail with reference to FIGS. 3B and 3C). The outer housing 132 has an outer peripheral wall 136, and includes a wide section 138 at the top, a narrow section 140 in the middle, and a tapering section 142 at the base. On opposite sides of the wide portion 138, there are finger hooks 144a, 144b. Each hook 144a, 144b includes an attachment portion 146a, 146b which is a planar element extending outwards from the peripheral wall 136 in a direction generally perpendicular to it. The plane of the planar elements is generally perpendicular to the longitudinal axis A. At the end of each attachment portion 146a, 146b is a winged portion 148a, 148b which is in the form of a concave rectangular piece of material attached at a central portion to an outer end of the respective attachment portion 146a, 146b. In use, as the user presses down on the handle portion 106 with their thumb, their index and middle fingers may rest in the regions 150a, 150b defined by the bottom surface of the attachment portions 146a, 146b and the bottom half of the winged portions, allowing them to exert more force on the handle 106 without their hand slipping. This will become clearer when the operation of the device is described with reference to FIGS. 3D and 3E.

At least part of the narrow portion 140 of the peripheral wall 136 is transparent or translucent, and in use, may have affixed thereto or printed thereon an identification code, as discussed previously in this application.

FIGS. 3B and 3C show the containment component 104 in an arrangement in which the inner mechanism 134 and the outer housing 132 are in the first relative configuration. The inner surface 152 of the outer housing 132 defines a generally cylindrical cavity. The internal base surface 154 of the outer housing includes a protrusion 156 having cylindrical symmetry about the central axis. The protrusion 156 includes a first frustoconical section 158 having a first outer frustoconical surface 159 and a second frustoconical section 160 having a second outer frustoconical surface 161, wherein the base of the second frustoconical section 160 is integrally formed with the upper surface of the first frustoconical section 158. The base of the second frustoconical section 160 has a smaller radius than the radius of the upper surface of the first frustoconical section 158, to define an upper annular surface 162. The portion of the base surface 154 excluding the protrusion 156 also defines an annular surface 164 which is parallel to the annular surface 162.

The internal diameter of the outer housing 132 is wide in the wide portion 138, narrower in the narrow section 140, and tapers in the tapering section 142 at the base. The internal surface 152 of the outer housing 132 in the wide portion 138 includes a circumferential ridge 166 which forms part of a locking mechanism discussed with reference to FIG. 3E.

The inner mechanism 134 of the containment component 104 is also generally cylindrical, and shares its axis with the outer housing 132. An outer diameter of the inner mechanism 132 is approximately equal to, or slightly smaller than an internal diameter of the outer housing 132 such that the inner mechanism 134 is able to fit inside the generally cylindrical cavity formed defined by the inner surface 152 of the outer housing 132. The outer surface 168 of the inner mechanism 134 is generally cylindrical and includes a tapering section 170 in which the diameter of the outer surface increases from a narrowest point 170n to a widest point 170w. After the widest portion 170w, the diameter of the outer surface 168 returns abruptly to the same as the diameter at the narrowest point 170n, thereby defining an annular abutment surface 172. The maximum diameter at the widest portion 170w is larger than the minimum internal diameter defined by the circumferential ridge 166 on the internal surface 152 of the wide portion 138 of the outer housing 132 of the containment component 104. Close to the bottom of the outer surface 168 of the inner mechanism 134 there are two circumferential flanges 174, 176, each having an outer diameter which is approximately equal to the internal diameter of the outer housing 132, so that the outer edges of the flanges 174, 176 contact the internal surface 152, thereby generating a friction force between the inner mechanism 134 and the outer housing 132, which acts to restrict relative axial motion between the two in the absence of an axial force having sufficient magnitude, discussed shortly.

The inner surface 178 of the inner mechanism 134 is also generally cylindrical, and includes a tapering portion 180. In the tapering portion, the internal diameter of the inner surface 178 of the inner mechanism decreases from a wide point 180w to a narrow point 180n, at which point the internal diameter returns abruptly to the wide value it was at point 180w. This defines an annular abutment surface 182. At its narrowest point, the internal diameter of the inner surface 178 of the inner mechanism is less than the outer diameter of the base plate 130.

The inner mechanism 134 includes a base plate 184 which is in the form of a disc of material located towards the bottom end of the inner mechanism 134. An outer circumferential region 186 (i.e. a region which lies outside a certain radius R1 of the base plate) includes a plurality of holes 188. The base surface 192, i.e. the one which faces downwards in the drawings, is generally flat. It also includes a cylindrical wall 194, having an outer surface 195, inner surface 197 defining a recess 196, and an annular end surface 199. The outer radius of the wall (i.e. the radius of the outer surface of the cylindrical wall 194) is less than or equal to the radius R1, such that none of the holes 188 extends through to the recess 196.

When the inner mechanism 134 is placed inside the outer housing 132 as shown in FIG. 3B, the annular end surface 199 of the wall 194 abuts the annular surface 162 of the protrusion 156.

The base surface 192 which is located outside the recess 196, the base surface 154 of the outer housing, the first outer frustoconical surface 159, and the lower part of the inner surface 178 of the inner mechanism 134 define a toroidal first reagent chamber 200 containing a first reagent 202.

The inner surface 197 of the wall 194 engages with, and preferably forms a seal with the second outer frustoconical surface 161. The base surface 192 within the recess 196, the inner surface 197, and an upper surface of the second frustoconical section 160 thus define a second reagent chamber 204, which contains a second reagent 206. The wall 194 acts as a barrier portion, isolating the first reagent chamber 200 from the second reagent chamber 204.

We now discuss the operation of the sample vessel 100 with reference to FIGS. 3D and 3E. Please note that only those features which are important for the operation of the sample vessel 100 are labelled in FIGS. 3D and 3E. Firstly, the test subject chews and sucks on the absorbent body 108 in order to saturate it with saliva. It will be appreciated that saliva is given as an example here only, and other bodily fluids may be used. The sample collection assembly 102 is then inserted into the cavity defined by the inner surface 178 of the inner mechanism 134 of the containment component 104. The sample collection assembly 102 is then lowered until a bottom surface of the absorbent body contacts the base plate 184 of the inner mechanism 134. At that point, by hooking their index and ring fingers under the finger hooks 144a, 144b, and pressing on handle 106 with their thumb, they compress the absorbent body 108 against the base plate 184. Compression of the absorbent body 108 causes the liquid with which it is saturated to flow through the holes 188 in the base plate 184 into the toroidal first reagent chamber 200, whereupon it may interact with the first reagent 202, thereby generating intermediate reagent 208 (or a fluid containing it). As shown in the zoomed in portion of FIG. 3D, when the handle 106 in sufficiently, the outer edge of the base plate 130 will slide past the tapering portion 180 (aided by the tapering shape), and become caught under the lip defined by the annular abutment surface 182. The abutment between the base plate 130 and the abutment surface 182 prevents the re-expansion of the absorbent body 108, effectively locking the sample collection assembly 102 relative to the inner mechanism 134. The sample vessel 100 may then be left so that the first reagent 202 can interact with the target species in the bodily fluid.

The next stage is shown in FIG. 3E. By pulling on the handle 106 (e.g. by placing the index and ring fingers under the wings 112a, 112b of the handle 106, the sample collection assembly 102 is no longer able to move out of the inner mechanism 134. Under the application of sufficient axial force to overcome the friction resulting from the contact between the outer edges of flanges 174, 176 and the inner surface 152 of the outer housing 132, the inner mechanism 134 is able to move axially within the outer housing 132. In the frame of reference where the outer housing 132 is stationary, the inner mechanism 134 (and the sample collection assembly 102, which is locked to it) move upwards. This causes the end surface 199 of the wall to disengage from annular abutment surface 162, meaning that the first reagent chamber 200 and the second reagent chamber 204 come into fluid communication with each other. This means that the second reagent 206 can tumble into the mixture 208 of bodily fluid and first reagent 202 in the toroidal first reagent chamber 200, so that a second interaction may take place. Then, once the second interaction is complete, and the fluid sample has been fully generated, the image may be captured using the image capture device 1002 through the transparent region discussed previously. Abutment between abutment surface 172 and the circumferential ridge 166 prevents the inner mechanism 134 from being fully removed.

We now discuss examples of the reagents which may be used in the present invention, in order to give rise to reactions which cause the desired changes in optical property. In preferred cases, a colour change of the fluid sample indicates the presence of a target species. Below, we provide more description of the biochemical aspects of this process.

The second reagent may include a component, an optical property of which changes in response to an interaction with a chemical entity, and a liposome encapsulating the chemical entity. In order to release the chemical entity, the first reagent may include a component which forms, generates, releases, or liberates a liposomal membrane destabilizing agent in the presence of the target species.

Specifically, the first reagent may comprise: (i) a first complex comprising: a liposome-destabilising agent capable of destabilising the liposomal membrane, and an aptamer specific for the liposome-destabilising agent, wherein the aptamer neutralises liposomal membrane-destabilisation by the liposome-destabilising agent; and (ii) a second complex comprising: an aptamer specific for the target species, hybridised to an antisense nucleic acid to the aptamer specific for the liposome-destabilising agent. Then, in the presence of the target species, the aptamer specific for the target molecule may bind to the target species, disrupting the second complex; the antisense nucleic acid to the aptamer specific for the liposome-destabilising agent liberated from the second complex binds to the aptamer specific for the liposome-destabilising agent, disrupting the first complex of (ii), thereby liberating the liposome-destabilizing agent. This process may take place in the first reagent chamber, and may take some time. Then, when exposed to the second reagent, the liposome-destabilising agent liberated from the first complex may destabilize the liposomal membrane of the liposome of, resulting in the release of the chemical entity, which can then interact with the component of the second reagent, thereby producing a change in optical property of the fluid sample.

In other similar case, the first reagent may include a single complex, comprising: a liposome-destabilising agent capable of destabilising the liposomal membrane of the liposome, and a composite aptamer, comprising: an aptamer specific for the liposome-destabilising agent, wherein the aptamer neutralises liposomal membrane-destabilisation by the liposome-destabilising agent, and an aptamer specific for the target species. In that case, in the presence of the target species
wherein in the presence of the target molecule: the aptamer specific for the target molecule may bind to the target molecule, disrupting of the complex, and the liposome-destabilising agent liberated from the complex may destabilize the liposomal membrane of the liposome, resulting in the release of the chemical entity which can then interact with the component of the second reagent, thereby producing a change in the optical property of the fluid sample.

The first reagent may further comprise a second liposome encapsulating antisense nucleic acid to the aptamer specific for the liposome-destabilising agent which binds to the aptamer specific for the liposome-destabilising agent, disrupting the complex. The liposomal membrane of the second liposome may be destabilised by release of the chemical entity from the (first, other) liposome. The liposome-destabilising agent may be lithocholic acid, and wherein the first liposome may be destabilised by lithocholic acid.

The chemical entity for producing a detectable signal may be an acidic species. The liposomal membrane of the second liposome may be destabilised by low pH.

The target species may be an antigen of SARS-CoV-2.

The methods of the present disclosure employ molecular displacement systems. The principles, design and application of nucleic acid strand-displacement systems are described e.g. in Simmel et al., Chem. Rev. (2019) 119(10): 6326-6369, Haley et al., Nature Comm. (2020) 11: 2562, Li et al., Sensors and Actuators B: Chemical (2017) 238: 626-632 and Wang et al., PNAS (2018) 115(52): E12182-E12191, all of which are hereby incorporated by reference in their entirety. Such systems have been employed in diagnostic applications to arrive at a result within minutes (see e.g. Wang et al., PNAS (2018) 115(52): E12182-E12191).

In cases of particular interest described herein, an aptamer for the RBD of SARS-CoV-2 spike protein is hybridised with antisense nucleic acid for a bile acid-specific aptamer forming a heteroduplex, and bile acid is separately bound by the bile acid-specific aptamer. If SARS-CoV-2 is present in a sample, the aptamer for the RBD of SARS-CoV-2 spike protein dissociates from the heteroduplex and binds to the RBD of SARS-CoV-2 spike protein. As a result, the bile acid-specific aptamer antisense nucleic acid is liberated and subsequently hybridises with the aptamer specific for the bile acid, liberating bile acid to induce destabilisation of the liposomal membrane of signal liposomes encapsulating an entity for producing a colour change in the reaction medium, having a liposomal membrane sensitive to destabilisation by bile acid. An amplification liposome encapsulating bile acid-specific aptamer antisense nucleic acid and having a liposomal membrane sensitive to destabilisation by release of the entity for producing a colour change in the reaction medium may also be provided, for amplifying the signal produced.

In further cases of particular interest described herein, a composite aptamer is provided comprising an aptamer for the RBD of SARS-CoV-2 spike protein, covalently linked to a bile acid-specific aptamer, along with the bile acid and signal liposomes encapsulating an entity for producing a colour change in the reaction medium, having a liposomal membrane sensitive to destabilisation by bile acid. The composite aptamer is only able to bind to the bile acid in the absence of the RBD of SARS-CoV-2 spike protein, such that when SARS-CoV-2 is present in a sample, the composite aptamer binds to the RBD of SARS-CoV-2 spike protein and liberates bile acid. The liberated bile acid destabilises signal liposomes, producing a colour change in the reaction medium. An amplification liposome encapsulating bile acid-specific aptamer antisense nucleic acid, and having a liposomal membrane sensitive to destabilisation by release of the entity for producing a colour change in the reaction medium may again be provided, for amplifying the signal produced.

In preferred cases, the signal liposome is sensitive to bile salt but resistant to low pH, and the entity for producing a colour change comprises acidic species, such that release of the entity from the signal liposome lowers the overall pH of the reaction medium; and the amplification liposome is sensitive to destabilisation by low pH. It will be appreciated that destabilisation of the amplification liposome releases more bile acid-specific aptamer antisense nucleic acid, liberating more bile acid and thus producing a feedback loop.

### Liposome Encapsulating a Chemical Entity for Producing a Detectable Signal

The articles and methods of the present disclosure employ liposomes. As used herein, a ‘liposome’ refers to a vesicle comprising a membrane formed by a lipid bilayer, surrounding an aqueous cavity. Liposomes according to the present disclosure may comprise a membrane formed by a phospholipid bilayer.

Liposomes and methods for the preparation of liposomes are described in Akbarzadeh et al., Nanoscale Res. Lett. (2013) 8(1): 102 and Rongen and Bennekom, J Immunol. Meth. (1997) 204: 105-133, both of which are hereby incorporated by reference in their entirety. Liposomes can be used to encapsulate chemical species, e.g. species present in the aqueous phase during liposome preparation.

The liposome may a chemical entity for producing a detectable signal. It will be appreciated that chemical entities include e.g. biomolecules such as nucleic acids, proteins, etc. The chemical entity may be any chemical entity suitable for producing a detectable signal, and which can also be encapsulated in a liposome. It will be appreciated in the context of the present application that the chemical entity produces a detectable signal on liberation from the liposome in which it is encapsulated, e.g. into the extra-liposomal space. The liposome encapsulating a chemical entity for producing a detectable signal may also be referred to herein as a ‘signal liposome’.

In some cases, the chemical entity encapsulated by the liposome may comprise or consist of a moiety for bringing about a detectable chemical change, e.g. a change in pH. That is, in some embodiments the detectable signal is a chemical change, e.g. a change in pH. For example, in some cases the chemical entity may comprise or consist of an acidic species or a basic species. In cases wherein the chemical entity comprises or consists of a moiety for bringing about a detectable chemical change, the articles and methods of the present disclosure may further employ reagents required for analysis of the detectable chemical change.

In particular embodiments, the chemical entity encapsulated by the liposome comprises or consists of an acidic species. In connection with such embodiments, liberation of such species following destabilisation of the liposome encapsulating the chemical entity preferably results in a detectable lowering of the pH of the medium into which the acidic species is released. The present disclosure contemplates the use of pH-sensitive reagents that change colour in response to a reduction in the pH following release of the chemical entity from the liposome, e.g. a colorimetric pH indicator.

In some embodiments the detectable signal may be an activity or property of the chemical entity. In some embodiments, the chemical entity may comprise or consist of a detectable moiety. In accordance with such embodiments, the methods may comprise detecting the detectable moiety.

A detectable moiety may be e.g. a fluorescent, luminescent, immuno-detectable, radiolabel, chemical, nucleic acid or polypeptide label. In some embodiments, the chemical entity may comprise or consist of a moiety having detectable activity, e.g. an enzymatic activity on a given substrate. Examples of chemical entities having detectable activity include e.g. horseradish peroxidase (HRP) and luciferase moieties. In embodiments wherein the chemical entity comprises or consists of a detectable moiety, the articles and methods of the present disclosure may further employ reagents required for analysis of the detectable activity. Detection techniques to be employed with such detectable moieties are well known to those of skill in the art and can be selected to correspond with the detectable moiety.

The present disclosure is concerned in particular with liposomes susceptible to inducible destabilisation. As used herein, ‘destabilisation’ of a liposome refers to disruption of the liposomal membrane such that its integrity is compromised, with the result that the contents of the liposome are able to diffuse freely to the extra-liposomal space. In the context of the liposome encapsulating a chemical entity for producing a detectable signal of the present disclosure, destabilisation of the liposome liberates the chemical entity for producing a detectable signal.

Destabilisation of a liposome encapsulating a chemical entity for producing a detectable signal according to the present disclosure is preferably chemically-inducible, e.g. by a liposome-destabilising agent described herein. Accordingly, in some embodiments, the liposome comprises a membrane susceptible to destabilisation by a liposome-destabilising agent.

It will be appreciated that the composition of the lipid bilayer of a liposome of the present disclosure may be selected in accordance with desired susceptibility to a liposome-destabilising agent described herein. For example, in any given embodiment of the articles and methods of the present disclosure, the composition of the membrane of the liposome encapsulating a chemical entity for producing a detectable signal according to the present disclosure is selected so as to be susceptible to destabilisation by the liposome-destabilising agent employed.

A given liposome can be evaluated for susceptibility to destabilisation by a given agent in a suitable in vitro assay. Such assays may include e.g. contacting a liposome encapsulating a detectable entity with the agent, and analysing the level of liberated (i.e. non-encapsulated) detectable entity after a period of time sufficient for destabilisation of the liposome by the agent to have occurred.

In such an assay, a liposome susceptible to destabilisation by the given agent/condition may be identified by the detection of an increase in the level of non-encapsulated (i.e. free, liberated) detectable entity, e.g. to greater than 1 times, e.g. one of greater than 2, 5, 10, 20, 30, 40, 50 or 100 times the level of liberated detectable entity detected in the absence of the agent/condition.

A liposome which is not susceptible to destabilisation by the given agent/condition may be identified by the detection of a level of non-encapsulated detectable entity which is similar to the level of the liberated detectable entity detected in the absence of the agent/condition. A ‘similar’ level of liberated detectable entity to a reference level may be ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, or ≥0.95 times and ≤1.1 times the reference level.

In preferred embodiments, the liposome encapsulating a chemical entity for producing a detectable signal according to the present disclosure is not susceptible to destabilisation by the chemical entity for producing a detectable signal. In preferred embodiments, the liposome is not susceptible to destabilisation as a consequence of an increase in the amount of non-encapsulated chemical entity for producing a detectable signal. In some embodiments, the liposome encapsulating a chemical entity for producing a detectable signal is not destabilised by low pH. The liposome encapsulating a chemical entity for producing a detectable signal may be referred to as a low pH-resistant liposome.

As used herein, ‘low pH’ refers to pH less than 7.0, e.g. one of pH≤6.5, ≤6.0, ≤5.5, ≤5.0, ≤4.5, ≤4.0, ≤3.5, ≤3.0, ≤2.5, ≤2.0, ≤1.5, ≤1.0 or ≤0.5.

In particular embodiments, a liposome encapsulating a chemical entity according to the present disclosure is susceptible to destabilisation by a bile acid. In some embodiments, the liposome is susceptible to destabilisation by a bile acid selected from lithocholic acid, cholic acid, deoxycholic acid, chenodeoxycholic acid, ursodeoxycholic acid, glycocholic acid, glycochenodeoxycholic acid, glycodeoxycholic acid, taurocholic acid, taurochenodeoxycholic acid and taurodeoxycholic acid. In some embodiments, the liposome is susceptible to destabilisation by lithocholic acid.

In some embodiments, a liposome encapsulating a chemical entity according to the present disclosure comprises a bilayer having the composition of a liposome described in Bulbake et al., Pharmaceutics (2017) 9(2): 12, which is hereby incorporated by reference in its entirety.

In particular embodiments, a liposome encapsulating a chemical entity according to the present disclosure comprises a bilayer having the composition of a liposome described in Roland and Wooley, Biochimica et Biophysica Acta (1980) 620: 400-409, which is hereby incorporated by reference in its entirety.

In some embodiments, a liposome encapsulating a chemical entity according to the present disclosure has a membrane comprising one or more (e.g. 2, 3, etc.) of the following: DL-3-dipalmitoyl phosphatidylcholine (DPPC), cholesterol, L-3-dimyristoyl phosphatidylcholine (DMPC), L-α-dilauroyl phosphatidylcholine (DLPC), L-α-distearoyl phosphatidylcholine (DSPC), L-α-dioleoyl phosphatidylcholine (DOPC), DL-3-dipalmitoyl phosphatidylethanolamine (DPPE), dicetylphosphate, egg phosphatidylcholine, stearylamine, L-α-dipalmitoyl phosphatidic acid (DPPA), hydrogenated soy phosphatidylcholine, dipalmitoylphosphatidylglycerol (DPPG), dioleoylphosphatidylserine (DOPS), palmitoyloleoylphosphatidylcholine (POPC), sphingomyelin, methoxy polyethylene glycol (MPEG), distearoyl snglycerophosphoethanolamine (DSPE), dimyristoyl phosphatidylglycerol (DMPG), distearoylphosphatidylglycerol (DSPG), cholesteryl sulphate, dierucoylphosphatidylcholine (DEPC), and dioleoly snglycerophophoethanolamine (DOPE).

In some embodiments, a liposome encapsulating a chemical entity according to the present disclosure has a membrane comprising one or more (e.g. 2, 3, etc.) of the following: DL-3-dipalmitoyl phosphatidylcholine (DPPC), cholesterol, L-3-dimyristoyl phosphatidylcholine (DMPC), L-α-dilauroyl phosphatidylcholine (DLPC), L-α-distearoyl phosphatidylcholine (DSPC), L-α-dioleoyl phosphatidylcholine (DOPC), DL-3-dipalmitoyl phosphatidylethanolamine (DPPE), dicetylphosphate, egg phosphatidylcholine, stearylamine, L-α-dipalmitoyl phosphatidic acid (DPPA).

In some embodiments, a liposome encapsulating a chemical entity according to the present disclosure has a membrane formed of DPPC and cholesterol, e.g. in a molar ratio of 7:2. In some embodiments, the liposome has a membrane formed of DMPC and cholesterol, e.g. in a molar ratio of 7:2. In some embodiments, the liposome has a membrane formed of DLPC and cholesterol, e.g. in a molar ratio of 7:2. In some embodiments, the liposome has a membrane formed of DSPC and cholesterol, e.g. in a molar ratio of 7:2. In some embodiments, the liposome has a membrane formed of DOPC and cholesterol, e.g. in a molar ratio of 7:2. In some embodiments, the liposome has a membrane formed of DPPE, cholesterol and dicetylphosphate, e.g. in a molar ratio of 7:2:1. In some embodiments, the liposome has a membrane formed of egg phosphatidylcholine, cholesterol and dicetylphosphate, e.g. in a ratio of 10:2:1 by weight. In some embodiments, the liposome has a membrane formed of egg phosphatidylcholine, cholesterol and stearylamine, e.g. in a ratio of 10:2:1 by weight. In some embodiments, the liposome has a membrane formed of DPPC and DPPA, e.g. in a ratio of 9:1 by weight.

### Liposome-Destabilising Agent

The articles and methods of the present disclosure employ liposome-destabilising agents. Liposome destabilising agents are characterised by their ability to disrupt the membranes of liposomes, resulting in liberation of their contents.

Liposome destabilisation by a liposome-destabilising agent according to the present disclosure may occur e.g. as a consequence of solubilisation of the liposomal membrane, or through the formation of pores or other defects in the liposomal membrane. A liposome-destabilising agent may be described as having lytic activity with respect to the liposome/liposomal membrane it destabilises.

In particular, the present disclosure is concerned with liposome-destabilising agents capable of destabilising liposomes encapsulating a chemical entity for producing a detectable signal according to the present disclosure.

It will be appreciated that the liposome-destabilising agent may be selected in accordance with the composition of the lipid bilayer of liposomes encapsulating a chemical entity for producing a detectable signal. For example, in any given embodiment of the articles and methods of the present disclosure, the selected liposome-destabilising agent has the ability to destabilise the membrane of the liposome encapsulating a chemical entity for producing a detectable signal.

A putative liposome-destabilising agent can be evaluated for the ability to destabilise a given liposome by analysis in a suitable in vitro assay. Such assays may include e.g. contacting a given liposome encapsulating a detectable entity with the putative liposome-destabilising agent, and analysing the level of liberated (i.e. non-encapsulated) detectable entity after a period of time sufficient for destabilisation of the liposome by the agent to have occurred.

In such an assay, a liposome-destabilising agent according to the present disclosure may result in an increase in the level of non-encapsulated (i.e. free, liberated) detectable entity to a level which is greater than 1 times, e.g. one of greater than 2, 5, 10, 20, 30, 40, 50 or 100 times the level of liberated detectable entity detected in the absence of the liposome-destabilising agent.

In some embodiments, the liposome-destabilising agent solubilises the liposomal membrane of liposomes susceptible to destabilisation by the liposome-destabilising agent. That is, in some embodiments the liposome-destabilising agent is a liposomal membrane-solubilising agent. Lipid membrane disruption by detergent solubilisation is described e.g. in Lichtenberg et al., Biophys J (2013) 105(2):289-99, which is hereby incorporated by reference in its entirety.

In some embodiments, the liposome-destabilising agent results in the formation of pores in the liposomal membrane of liposomes susceptible to destabilisation by the liposome-destabilising agent. That is, in some embodiments the liposome-destabilising agent is a liposomal membrane pore-forming agent.

In some embodiments, the liposome-destabilising agent is a membrane-lytic peptide. The structure and function of membrane-lytic peptides is described e.g. in Beckinger, Crti. Rev. Plant Sci. (2004) 23(3): 271-292 and Shai Biochimica et Biophysica Acta—Biomembranes (2004) 1462(1-2): 55-70, both of which are hereby incorporated by reference in their entirety. In some embodiments, the liposome-destabilising agent is a membrane-lytic peptide described in Beckinger, Crti. Rev. Plant Sci. (2004) 23(3): 271-292 or Shai Biochimica et Biophysica Acta—Biomembranes (2004) 1462(1-2): 55-70.

In some embodiments, the liposome-destabilising agent is a membrane-lytic peptide described in Beckinger and Lohner Biochimica et Biophysica Acta—Biomembranes (2006) 1758(9): 1529-1539, which is hereby incorporated by reference in its entirety. In some embodiments, the liposome-destabilising agent is an amphipathic cationic peptide. In some embodiments, the liposome-destabilising agent is a magainin, cecropin, dermaseptin, δ-lysin or melittin.

In some embodiments, the liposome-destabilising agent is a membrane-lytic peptide described in dos Santos Cabrera Langmuir (2011) 27(17) 10805-10813, which is hereby incorporated by reference in its entirety. In some embodiments, the liposome-destabilising agent is a mastoparan. In some embodiments, the liposome-destabilising agent is polybia-MP-1 (MP-1), N2-polybia-MP-1 (N-MP-1) or mastoparan X.

In some embodiments, the liposome-destabilising agent is a bile acid. Bile acids have been shown to increase permeability of phospholipid membranes. In some embodiments, the liposome-destabilising agent is a bile acid selected from lithocholic acid, cholic acid, deoxycholic acid, chenodeoxycholic acid, ursodeoxycholic acid, glycocholic acid, glycochenodeoxycholic acid, glycodeoxycholic acid, taurocholic acid, taurochenodeoxycholic acid and taurodeoxycholic acid. In some embodiments, the liposome-destabilising agent is lithocholic acid.

In accordance with the methods and articles of the present disclosure, it will be appreciated that the liposome-destabilising agent is preferably provided saturated with neutralising aptamer specific for the liposome-destabilising agent, such that there is no or minimal destabilisation of the liposome encapsulating a chemical entity for producing a detectable signal in the absence of the relevant target molecule.

### Aptamers

The articles and methods of the present disclosure employ aptamers. As used herein, an “aptamer” refers to a nucleic acid which binds (e.g. with high affinity and specificity) to a target molecule. A target molecule may be e.g. a peptide/polypeptide, glycoprotein, lipoprotein, glycan, glycolipid, lipid, or small molecule. Aptamer structure and properties are reviewed, for example, in Zhou and Rossi, Nat Rev Drug Discov. (2017) 16(3): 181-202.

Aptamers according to the present disclosure may comprise or consist of polymers of deoxyribonucleotides or ribonucleotides. Aptamers contemplated herein include DNA polynucleotides and RNA polynucleotides, and generally comprise ˜2-100 nucleotides. Methods for synthesising DNA and RNA aptamers are well known in the art, and are described, for example, in Roy and Caruthers, Molecules (2013) 18:14268-14284, which is hereby incorporated by reference in its entirety. Suitable methods include synthesis according to the phosphoramidite method.

Aptamers may comprise, or consist of, RNA or DNA, and may be single-stranded or double-stranded. In preferred embodiments, aptamers according to the present disclosure consist of ssDNA. Aptamers may have secondary or tertiary structure and, thus, may be able to fold into diverse and intricate molecular structures.

Aptamers may comprise chemically modified nucleotides or nucleosides, for example in which the sugar and/or phosphate and/or base is chemically modified. Such modifications may improve the stability of the aptamer or make the aptamer more resistant to degradation. The aptamers of the present disclosure may include chemical modifications as described herein such as a chemical substitution at a sugar position, a phosphate position, and/or a base position of the nucleic acid including, for example, incorporation of a modified nucleotide, incorporation of a capping moiety (e.g. 3′ capping), conjugation to a high molecular weight, non-immunogenic compound (e.g. polyethylene glycol (PEG)), conjugation to a lipophilic compound, substitutions in the phosphate backbone. Base modifications may include 5-position pyrimidine modifications, modifications at exocyclic amines, substitution of 4-thiouridine, substitution of 5-bromo- or 5-iodo-uracil, backbone modifications. Sugar modifications may include 2′-amine nucleotides (2′-NH₂), 2′-fluoro nucleotides (2′-F), and 2′-O-methyl (2′-OMe) nucleotides. A wide range of nucleotide, nucleoside, base and phosphate modifications are known to those of ordinary skill in the art, e.g. as described in Eaton et al., Bioorganic & Medicinal Chemistry (1997) 5 (6): 1087-1096.

Aptamers may be synthesised by methods which are well known to the skilled person. For example, aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D.; Biernat, J.; McManus, J.; Köster, H., Nucleic Acids Res. (1984), 12, 4539; and Beaucage, S. L.; Lyer, R. P., Tetrahedron (1992)48 (12): 2223).

Aptamers can be selected in vitro from libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al., (2010) Aptamer-based multiplexed proteomic technology for biomarker discovery. PLOS ONE 5(12):e15004). Applying the SELEX and the SOMAmer technology includes for instance adding functional groups that mimic amino acid side chains to expand the aptamer's chemical diversity. As a result, high affinity aptamers for a protein may be enriched and identified.

Aptamers according to the present disclosure may be monospecific, i.e. having high affinity and specificity for a single target molecule, and can be thought of as the nucleic acid equivalent of monoclonal antibodies. As with monoclonal antibodies, they may be useful in virtually any situation in which target binding is required, including use in therapeutic and diagnostic applications, in vitro or in vivo. In vitro diagnostic applications may include use in detecting the presence or absence of a target molecule.

An aptamer according to the present disclosure may be capable of specific binding to a target molecule. As used herein, “specific binding” refers to binding which is selective for the target molecule, and which can be discriminated from non-specific binding to non-target molecule. An aptamer that binds specifically to a target molecule preferably binds the target with greater affinity, and/or with greater duration than it binds to other, non-target molecules.

The ability of a given aptamer to bind specifically to a given target molecule can be determined by analysis according to methods known in the art, such as by microscale thermophoresis (MST), e.g. as described in Breitsprecher et al., Methods Mol Biol (2016) 1380: 99-111 (hereby incorporated by reference in its entirety), or as described in the Examples herein. Through such analysis, binding to a given molecule can be measured and quantified. In some embodiments, the binding may be the response detected in a given assay.

In some embodiments, the extent of binding of the aptamer to a non-target molecule is less than about 10% of the binding to the target molecule as measured using the given assay. Binding specificity may be reflected in terms of binding affinity, where the aptamer binds with a dissociation constant (Kd) that is at least 0.1 order of magnitude (i.e. 0.1×10ⁿ, where n is an integer representing the order of magnitude) greater than the Kd of the antigen-binding molecule towards a non-target molecule. This may optionally be one of at least 0.2, 0.3, 0.4, 0.5, 0.6, 0.7, 0.8, 0.9, 1.0, 1.5, or 2.0 orders of magnitude.

In some embodiments, an aptamer according to the present disclosure binds to its target with an affinity in the micromolar range, i.e. K_{D}=9.9×10⁻⁴ to 1×10⁻⁶ M. In some embodiments, an aptamer binds to its target with sub-micromolar affinity, i.e. K_{D}<1×10⁻⁶ M. In some embodiments, an aptamer binds to its target with an affinity in the nanomolar range, i.e. K_{D}=9.9×10⁻⁷ to 1×10⁻⁹ M. In some embodiments, an aptamer binds to its target with sub-nanomolar affinity, i.e. K_{D}<1×10⁻⁹ M. In some embodiments, an aptamer binds to its target with an affinity in the picomolar range, i.e. K_{D}=9.9×10⁻¹⁰ to 1×10⁻¹² M. In some embodiments, an aptamer binds to its target with sub-picomolar affinity, i.e. K_{D}<1×10⁻¹² M.

In aspects and embodiments of the present disclosure, aptamers according to the present disclosure display binding to a target molecule of interest in accordance with diagnostic and/or prognostic application of the methods and articles of the disclosure. That is, some aspects and embodiments of the invention employ aptamers for the detection of a target molecule of interest, e.g. in a sample containing/suspected to contain the target molecule.

A target molecule may be any molecule of interest to be detected. In some embodiments, the target molecule is a molecule whose presence/expression/activity, or whose upregulated expression/activity, is positively associated with a disease or disorder (e.g. an infectious disease, a cancer or an autoimmune disease). In some embodiments, the target molecule is expressed by, or encoded by nucleic acid of, an infectious agent. In some embodiments, the target molecule is expressed by cells infected with an infectious agent. An infectious agent may be e.g. a viral, bacterial, fungal or protozoal pathogen. In some embodiments, the target molecule is expressed by cells of a cancer. In some embodiments, the target molecule is expressed by an autoimmune effector cell.

In embodiments of particular interest, the aptamer specific for a target molecule is specific for an antigen of a virus.

In some embodiments, the target molecule is an antigen of a SARSr-CoV. SARSr-CoV is a species of coronavirus of the genus Betacoronavirus and subgenus Sarbecoronavirus that infects humans, bats and certain other mammals. It is an enveloped positive-sense single-stranded RNA virus. Two strains of SARSr-CoV have caused serious outbreaks of severe respiratory diseases in humans: SARS-CoV, which caused an outbreak of severe acute respiratory syndrome (SARS) between 2002 and 2003, and SARS-CoV-2, which has caused the coronavirus disease 2019 (CoVID-19) pandemic. There are a large number of strains of SARSr-CoV known only to infect non-human species; bats are a major reservoir of many strains of SARS-related coronaviruses. In some embodiments in accordance with the various aspects of the present disclosure, the SARS-CoV is a strain of SARSr-CoV that causes coronavirus disease in humans.

In some embodiments, the target molecule is an antigen of SARS-CoV-2. As used herein, “SARS-CoV-2” refers to the SARSr-CoV having the nucleotide sequence of GenBank: MN996527.1 (“Severe acute respiratory syndrome coronavirus 2 isolate WIV02, complete genome”), reported in Zhou et al., Nature (2020) 579: 270-273, and encompasses variants thereof having a nucleotide sequence with at least 85% sequence identity (e.g. one of at least 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or greater sequence identity) to the nucleotide sequence of GenBank: MN996527.1. The SARS-CoV-2 genome encodes four major structural proteins: the spike (S) protein, the envelope (E) protein, the membrane (M) protein, and the nucleocapsid (N) protein. In some embodiments, the target molecule is SARS-CoV-2 spike protein or a fragment thereof.

Aptamers to SARS-CoV-2 antigens are described e.g. in Torabi et al., Mol Cell Probes. (2020) October; 53: 101636 (see in particular Table 1) and Song et al., Anal. Chem. (2020) 92(14): 9895-9900, both of which are hereby incorporated by reference in their entirety.

In some embodiments the target molecule is SARS-CoV-2 spike protein, or a fragment thereof. In some embodiments, the target molecule is the S1 subunit of SARS-CoV-2 spike protein, or a fragment thereof. In some embodiments, the target molecule is the RBD of SARS-CoV-2 spike protein, or a fragment thereof.

In some embodiments, the aptamer specific for a target molecule is, or is derived from, a SARS-CoV-2 spike protein RBD-binding aptamer described in Song et al., Anal. Chem. (2020) 92(14): 9895-9900. In some embodiments the aptamer is, or is derived from, an aptamer selected from CoV2-RBD-1C, CoV2-RBD-1, CoV2-RBD-4 and CoV2-RBD-4C described in Song et al., Anal. Chem. (2020) 92(14): 9895-9900. In some embodiments the aptamer is, or is derived from, CoV2-RBD-1C described in Song et al., Anal. Chem. (2020) 92(14): 9895-9900. As used herein, an aptamer which is “derived from” a reference aptamer comprises or consists of a nucleotide sequence having at least 80% sequence identity (e.g. one of at least 81%, 82%, 83%, 84%, 85%, 86%, 87%, 88%, 89%, 90%, 91%, 92%, 93%, 94%, 95%, 96%, 97%, 98%, 99% or greater sequence identity) to the nucleotide sequence of the reference aptamer.

In some embodiments, binding of the aptamer specific for a target molecule to its target molecule disrupts a complex comprising an aptamer specific for the target molecule hybridised to an antisense nucleic acid to the aptamer specific for the liposome-destabilising agent. In some embodiments, binding of the aptamer specific for a target molecule to its target molecule displaces the antisense nucleic acid to the aptamer specific for a liposome-destabilising agent from a complex comprising an aptamer specific for the target molecule hybridised to an antisense nucleic acid to the aptamer specific for a liposome-destabilising agent. That is, in some embodiments the effect of binding of the aptamer specific for a target molecule to its target molecule is to liberate the antisense nucleic acid to the aptamer specific for a liposome-destabilising agent from the complex.

In some embodiments, the target molecule is an inhibitor of interaction between the aptamer specific for a target molecule and the antisense nucleic acid to the aptamer specific for a liposome-destabilising agent. In some embodiments, the target molecule is a competitive inhibitor of interaction between the aptamer specific for a target molecule and the antisense nucleic acid to the aptamer specific for a liposome-destabilising agent. In some embodiments, the target molecule is an allosteric inhibitor of interaction between the aptamer specific for a target molecule and the antisense nucleic acid to the aptamer specific for a liposome-destabilising agent.

In some embodiments, the ability of the aptamer specific for a target molecule to bind to the antisense nucleic acid to the aptamer specific for a liposome-destabilising agent is conditional on the aptamer specific for a target molecule not being bound to its target molecule. In some embodiments, a complex comprising the aptamer specific for a target molecule and its target molecule is not able to bind to the antisense nucleic acid to the aptamer specific for a liposome-destabilising agent. In some embodiments, binding of the aptamer specific for a target molecule to its target molecule removes the site through which the antisense nucleic acid to the aptamer specific for a liposome-destabilising agent hybridises with the aptamer specific for a target molecule.

In preferred embodiments, the aptamer specific for a target molecule binds its target molecule with greater affinity than the affinity with which it binds to the antisense nucleic acid to the aptamer specific for a liposome-destabilising agent.

Aspects and embodiments of the present disclosure also employ aptamers specific for liposome-destabilising agents, e.g. a liposome-destabilising agent as described hereinabove.

In embodiments of particular interest, the aptamer specific for a liposome-destabilising agent is specific for a bile acid. In some embodiments, the aptamer is specific for a bile acid selected from lithocholic acid, cholic acid, deoxycholic acid, chenodeoxycholic acid, ursodeoxycholic acid, glycocholic acid, glycochenodeoxycholic acid, glycodeoxycholic acid, taurocholic acid, taurochenodeoxycholic acid and taurodeoxycholic acid. In some embodiments, the aptamer is specific for lithocholic acid.

In preferred embodiments, aptamers specific for liposome-destabilising agents in accordance with the present disclosure preferably reduce/inhibit the ability of liposome-destabilising agent to destabilise the liposomal membrane. Such aptamers may be described as being antagonists of liposome destabilisation by the liposome-destabilising agent.

In further preferred embodiments, an aptamer specific for a liposome-destabilising agent according to the present disclosure neutralises liposome membrane destabilisation by the liposome-destabilising agent. Aptamers which neutralise liposomal membrane destabilisation by a liposome-destabilising agent may substantially inhibit or completely prevent liposome membrane destabilisation by the liposome-destabilising agent.

Aptamers can be analysed for their ability to inhibit/reduce/neutralise liposome membrane destabilisation by a given liposome-destabilising agent in a suitable in vitro assay. For example, the liposome-destabilising agent can be incubated with a molar excess of the aptamer, and the liposome-destabilising agent:aptamer mixture can subsequently be applied to liposomes encapsulating a detectable entity, and having a membrane destabilised by the given agent.

In such an assay, aptamers which reduce/inhibit liposomal membrane destabilisation by the liposome-destabilising agent can be identified by determination of a reduction in the level of liberated detectable entity as compared to the level detected where the liposome-destabilising agent is applied to the liposomes in the absence of the aptamer (or in the presence of an aptamer which does not bind to the liposome-destabilising agent).

In such assays, aptamers which reduce/inhibit liposomal membrane destabilisation by a given liposome-destabilising agent may reduce the level of liberated detectable entity to a level which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of liberated detectable entity detected in the absence of the aptamer.

In such assays, aptamers which neutralise liposomal membrane destabilisation by a given liposome-destabilising agent may reduce the level of liberated detectable entity to a level which is less than 0.3 times e.g. one of ≤0.25 times, ≤0.2 times, ≤0.15 times, ≤0.1 times, ≤0.05 times, or ≤0.01 times the level of liberated detectable entity detected in the absence of the aptamer. That is, the aptamer may inhibit 70% or greater, e.g. one of ≥75%, ≥80%, ≥90%, ≥95% or ≥99% of liposomal membrane destabilisation by the liposome-destabilising agent detected in the absence of the aptamer.

Aptamers which neutralise liposomal membrane destabilisation by the liposome-destabilising agent can be identified by determination of a level of liberated detectable entity which is similar to the level of detectable entity detected in the absence of the liposome-destabilising agent. A ‘similar’ level of liberated detectable entity to a reference level may be ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, or ≥0.95 times and ≤1.1 times the reference level.

In some embodiments, the aptamer specific for a liposome-destabilising agent binds to the liposome-destabilising agent in a region required for liposomal membrane destabilisation by the liposome-destabilising agent. In some embodiments, the aptamer inhibits/reduces/prevents interaction between the liposome-destabilising agent and the liposomal membrane it is capable of destabilising. In some embodiments, the aptamer binds to a region of the liposome-destabilising agent through which the liposome-destabilising agent contacts the liposomal membrane it is capable of destabilising. In some embodiments, the aptamer is an inhibitor of liposomal membrane destabilisation by the liposome-destabilising agent. In some embodiments, the aptamer is a competitive inhibitor of liposomal membrane destabilisation by the liposome-destabilising agent. In some embodiments, the aptamer is an allosteric inhibitor of liposomal membrane destabilisation by the liposome-destabilising agent.

The region of a molecule to which an aptamer binds can be determined by the skilled person using various methods known in the art, including X-ray co-crystallography analysis of aptamer:target complexes, and hydrogen-deuterium exchange mass spectrometry.

The ability of an aptamer to inhibit interaction between two factors can be determined for example by analysis of interaction in the presence of, or following incubation of one or both of the interaction partners with, the aptamer. An aptamer which is capable of inhibiting a given interaction (e.g. between a liposome-destabilising agent and the liposomal membrane it is capable of destabilising) is identified by the observation of a reduction/decrease in the level of interaction between the interaction partners in the presence of—or following incubation of one or both of the interaction partners with—the aptamer, as compared to the level of interaction observed in the absence of the aptamer (or in the presence of an appropriate control aptamer).

Aptamers to bile acid are described e.g. in Kato et al., Nucleic Acids Res. (2000) 28(9): 1963-1968, which is hereby incorporated by reference in its entirety.

In some embodiments, the aptamer specific for a liposome-destabilising agent is, or is derived from, an aptamer described in Kato et al., Nucleic Acids Res. (2000) 28(9): 1963-1968. In some embodiments the aptamer is, or is derived from, an aptamer shown in Table 1(a) of Kato et al., Nucleic Acids Res. (2000) 28(9): 1963-1968. In some embodiments the aptamer is, or is derived from, ch9-48 described in Kato et al., Nucleic Acids Res. (2000) 28(9): 1963-1968.

In some embodiments, a complex comprising the aptamer specific for a liposome-destabilising agent and the liposome-destabilising agent for which it is specific is disrupted by an antisense nucleic acid to the aptamer specific for a liposome-destabilising agent. In some embodiments, the aptamer specific for a liposome-destabilising agent is displaced from a complex comprising the aptamer specific for a liposome-destabilising agent and the liposome-destabilising agent for which it is specific by an antisense nucleic acid to the aptamer specific for a liposome-destabilising agent. That is, in some embodiments the aptamer specific for a liposome-destabilising agent is liberated from a complex comprising the aptamer specific for a liposome-destabilising agent and the liposome-destabilising agent for which it is specific by binding of an antisense nucleic acid to the aptamer specific for a liposome-destabilising agent to the aptamer specific for a liposome-destabilising agent.

In some embodiments, interaction between the aptamer specific for a liposome-destabilising agent and the liposome-destabilising agent for which it is specific is inhibited by an antisense nucleic acid to the aptamer specific for a liposome-destabilising agent. In some embodiments, interaction between the aptamer specific for a liposome-destabilising agent and the liposome-destabilising agent for which it is specific is competitively inhibited by an antisense nucleic acid to the aptamer specific for a liposome-destabilising agent.

In some embodiments, the ability of the aptamer specific for a liposome-destabilising agent to bind to the liposome-destabilising agent for which it is specific is conditional on the aptamer specific for a liposome-destabilising agent not being bound to an antisense nucleic acid to the aptamer specific for a liposome-destabilising agent. In some embodiments, a complex comprising the aptamer specific for a liposome-destabilising agent and an antisense nucleic acid to the aptamer specific for a liposome-destabilising agent is not able to bind to the liposome-destabilising agent for which the aptamer is specific. In some embodiments, binding of the aptamer specific for a liposome-destabilising agent to an antisense nucleic acid to the aptamer specific for a liposome-destabilising agent removes the site through which the aptamer specific for a liposome-destabilising agent binds to the liposome-destabilising agent for which it is specific.

In preferred embodiments, the aptamer specific for a liposome-destabilising agent binds to an antisense nucleic acid to the aptamer specific for a liposome-destabilising agent with greater affinity than the affinity with which it binds to the liposome-destabilising agent for which it is specific.

In some aspects and embodiments, the present disclosure provides an aptamer comprising: (a) an aptamer specific for a liposome-destabilising agent, and (b) an aptamer specific for a target molecule. That is, the disclosure provides a composite, multispecific aptamer comprising aptamers according to (a) and (b). It will be appreciated that the aptamer according to (a) is preferably an aptamer specific for a liposome-destabilising agent as described herein, and similarly that the aptamer according to (b) is preferably an aptamer specific for a target molecule as described herein.

The design and production of multispecific aptamer molecules is described e.g. in Vorobyeva et al., Molecules (2016) 21(12): 1613, which is hereby incorporated by reference in its entirety.

Aptamers according to (a) and (b) may be covalently or non-covalently linked to one another. In some embodiments, aptamers according to (a) and (b) are covalently associated with one another. For example, the aptamers may be connected via a phosphodiester bond. In some embodiments, aptamers (a) and (b) may be comprised in the same polynucleotide. In some embodiments, aptamers (a) and (b) are provided in tandem in the sequence of the polynucleotide.

In some embodiments, aptamers (a) and (b) are connected in the same polynucleotide by one or more (e.g. 2 to 50, 5-40, 10 to 30, or 15 to 20) nucleotides forming a linker sequence between the aptamers. The linker may comprise or consist of an oligo(A), oligo(U) or oligo(T) sequence. The nucleotide sequence, both in terms of its length and base composition, may be selected as appropriate in accordance with the desired binding characteristics of the aptamers (a) and (b).

Alternatively, aptamers (a) and (b) may be connected by a hydrocarbon linker, e.g. a polyethylene glycol (PEG) linker. PEG linkers may comprise one or more hexaethyleneglycol residues joined by phosphodiester bonds.

In preferred embodiments, a composite aptamer (i.e. an aptamer comprising: (a) an aptamer specific for a liposome-destabilising agent, and (b) an aptamer specific for a target molecule) according to the present disclosure comprises a structure such that:
(i) the composite aptamer binds through the aptamer of (a) to the liposome-destabilising agent for which it is specific in the absence of the target molecule for the aptamer of (b); and
(ii) the composite aptamer is not able to bind through the aptamer of (a) to the liposome-destabilising agent for which it is specific when the aptamer of (b) is bound to its target molecule.

The composite aptamer may be thought of as a ‘molecular trebuchet’, comprising a payload-bearing aptamer specific for the liposome-destabilising agent, and a sensor aptamer specific for the target molecule. Binding of the sensor aptamer to the target molecule disrupts the structure required for the payload-bearing aptamer to retain the liposome-destabilising agent.

The design and production of aptamers having such conditional binding properties is described e.g. in Vorobyeva et al., Molecules (2016) 21(12): 1613 and Tang et al., J. Am. Chem. Soc. (2008) 130(34): 11268-11269, both of which are hereby incorporated by reference in their entirety.

In some embodiments, binding of the aptamer of (b) to its target molecule inhibits/prevents/disrupts binding of the aptamer of (a) to the liposome-destabilising agent for which it is specific.

Binding of the target molecule to the composite aptamer through the aptamer of (b) may result in a change in the structure of the composite aptamer, inhibiting/preventing association between the composite aptamer and the liposome-destabilising agent for which the aptamer of (a) is specific.

In some embodiments, binding of the aptamer of (b) to its target molecule disrupts the complex formed between the composite aptamer and the liposome-destabilising agent for which the aptamer of (a) is specific.

In some embodiments, the target molecule for the aptamer of (b) displaces the liposome-destabilising agent from the complex formed between the composite aptamer and the liposome-destabilising agent for which the aptamer of (a) is specific. The effect is that binding of the target molecule to the composite aptamer liberates the liposome-destabilising agent from such complexes.

In some embodiments, the target molecule for the aptamer of (b) is an inhibitor of interaction between the aptamer of (a) and the liposome-destabilising agent for which it is specific. In some embodiments, the target molecule for the aptamer of (b) is a competitive inhibitor of interaction between the aptamer of (a) and the liposome-destabilising agent for which it is specific. In some embodiments, the target molecule for the aptamer of (b) is an allosteric inhibitor of interaction between the aptamer of (a) and the liposome-destabilising agent for which it is specific.

In some embodiments, the ability of the composite aptamer to bind to the liposome-destabilising agent for which the aptamer of (a) is specific is conditional on the composite aptamer not being bound to the target molecule for the aptamer of (b). In some embodiments, a complex comprising the composite aptamer and the target molecule for the aptamer of (b) is not able to bind to the liposome-destabilising agent for which the aptamer of (a) is specific. In some embodiments, binding of the target molecule for the aptamer of (b) to the composite aptamer removes the binding site for the liposome-destabilising agent for which the aptamer of (a) is specific.

In preferred embodiments, the composite aptamer binds to the target molecule for the aptamer of (b) with greater affinity than the affinity with which it binds to the composite aptamer to the liposome-destabilising agent for which the aptamer of (a) is specific.

Aptamers and nucleic acids can be evaluated for the properties described herein e.g. using appropriate in vitro assays for investigating interaction between nucleic acids, and/or between aptamers and their targets. Such assays may e.g. involve analysis by microscale thermophoresis (MST), which is described e.g. in Breitsprecher et al., Methods Mol Biol (2016) 1380: 99-111 (incorporated by reference hereinabove).

In some embodiments, aptamers according to the present disclosure may comprise one or more additional nucleotides in addition to the target-binding sequence. For example, an aptamer described herein may comprise 1, 2, 3, 4, 5, 6, 7, 8, 9, 10, 11, 12, 13, 14, 15, 16, 17, 18, 19 or 20 nucleotides at one or both end(s) of the target-binding sequence.

In some embodiments, aptamers according to the present disclosure may additionally comprise a detectable moiety. By way of illustration, in Example 2 herein an aptamer to SARS CoV-2 spike protein S1 subunit comprises a fluorescein label. The detectable moiety is useful for detection the aptamer and complexes (e.g. aptamer:target complexes) comprising the aptamer.

In some embodiments, a detectable moiety is selected from a fluorescent label, phosphorescent label, luminescent label, immuno-detectable label (e.g. an epitope tag), radiolabel, chemical, nucleic acid or enzymatic label. The aptamer molecule may be covalently or non-covalently labelled with the detectable moiety. Techniques for labelling nucleic acids such as aptamers with detectable moieties are well known to the skilled person, and are described e.g. in Klöcker et al., “Covalent labelling of nucleic acids”, Chem. Soc. Rev., 2020 (Advance Article), which is hereby incorporated by reference in its entirety.

Fluorescent labels include e.g. fluorescein, rhodamine, allophycocyanin, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, ThuliumI¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers. Enzymatic labels include e.g. peroxidase, alkaline phosphatase, glucose oxidase, beta-galactosidase and luciferase.

### Antisense Nucleic Acid to the Aptamer Specific for a Liposome-Destabilising Agent

Aspects and embodiments of the present disclosure relate to an antisense nucleic acid to the aptamer specific for the liposome-destabilising agent. It will be appreciated that the antisense nucleic acid to the aptamer specific for a liposome-destabilising agent binds to (i.e. hybridises with) the aptamer specific for the liposome-destabilising agent through complementary base pairing.

The nucleic acid may comprise, or consist of, RNA or DNA. In preferred embodiments, the nucleic acid consists of ssDNA.

Herein, an antisense nucleic acid to a given nucleic acid (e.g. an aptamer) is capable of hybridising to the given nucleic acid through complementary base pairing. In some embodiments, the antisense nucleic acid comprises or consists of a nucleotide sequence having high sequence identity (e.g. 80% or greater sequence identity) to the reverse complement of part or all of the nucleotide sequence of the given nucleic acid. In accordance with such embodiments, part of the nucleotide sequence of the given nucleic acid may be e.g. 20% or more (e.g. one of at least 25%, 30%, 35%, 40%, 45%, 50%, 55%, 60%, 65%, 70%, 75%, 80%, 85%, 90%, 95% or more) of the nucleotide sequence of the given nucleic acid.

In some embodiments, an antisense nucleic acid to a given nucleic acid (e.g. an aptamer) comprises or consists of a nucleotide sequence having at least 80% sequence identity (e.g. one of at least 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of part or all of the nucleotide sequence of the given nucleic acid.

Accordingly, in some embodiments an antisense nucleic acid to the aptamer specific for a liposome-destabilising agent comprises or consists of a nucleotide sequence having at least 80% sequence identity (e.g. one of at least 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of the nucleotide sequence of the aptamer specific for the liposome-destabilising agent.

In some embodiments, the antisense nucleic acid to the aptamer specific for a liposome-destabilising agent additionally comprises a nucleotide sequence having complementarity to the aptamer specific for the target molecule. The nucleotide sequence having complementarity to the aptamer specific for the target molecule may function as a toehold sequence as described hereinbelow, providing for hybridisation between the antisense nucleic acid to the aptamer specific for a liposome-destabilising agent and the aptamer specific for the target molecule.

In some embodiments, binding of the antisense nucleic acid to the aptamer specific for a liposome-destabilising agent to the aptamer specific for a liposome-destabilising agent disrupts a complex comprising the aptamer specific for a liposome-destabilising agent and the liposome-destabilising agent. In some embodiments, binding of the antisense nucleic acid to the aptamer specific for a liposome-destabilising agent to the aptamer specific for a liposome-destabilising agent displaces the liposome-destabilising agent from a complex comprising the aptamer specific for a liposome-destabilising agent and the liposome-destabilising agent. That is, in some embodiments the effect of binding of the antisense nucleic acid to the aptamer specific for a liposome-destabilising agent to the aptamer specific for a liposome-destabilising agent is to liberate the liposome-destabilising agent from the complex.

In some embodiments, the antisense nucleic acid to the aptamer specific for a liposome-destabilising agent is an inhibitor of interaction between the aptamer specific for a liposome-destabilising agent and the liposome-destabilising agent. In some embodiments, the antisense nucleic acid to the aptamer specific for a liposome-destabilising agent is a competitive inhibitor of interaction between the aptamer specific for a liposome-destabilising agent and the liposome-destabilising agent.

In some embodiments, the ability of the aptamer specific a liposome-destabilising agent to bind to the antisense nucleic acid to the aptamer specific for a liposome-destabilising agent is not conditional on the aptamer specific for a liposome-destabilising agent not being bound to the liposome-destabilising agent. In some embodiments, a complex comprising the aptamer specific for a liposome-destabilising agent and the liposome-destabilising agent is able to bind to the antisense nucleic acid to the aptamer specific for a liposome-destabilising agent. In some embodiments, binding of the aptamer specific for a liposome-destabilising agent to the liposome-destabilising agent does not remove the site through which the antisense nucleic acid to the aptamer specific for a liposome-destabilising agent hybridises with the aptamer specific for a liposome-destabilising agent.

In preferred embodiments, the antisense nucleic acid to the aptamer specific for a liposome-destabilising agent binds to the aptamer specific for a liposome-destabilising agent with greater affinity than the affinity with which the aptamer specific for a liposome-destabilising agent binds to the liposome-destabilising agent.

In accordance with the methods and articles of the present disclosure, it will be appreciated that the antisense nucleic acid to the aptamer specific for a liposome-destabilising agent is preferably provided saturated with aptamer specific for the target molecule, such that there is no or minimal disruption of complexes comprising the liposome-destabilising agent and aptamer specific for the liposome-destabilising agent in the absence of the relevant target molecule.

### Complexes

Aspects and embodiments of the present disclosure concern complexes, e.g. complexes comprising nucleic acids (e.g. aptamers, antisense nucleic acids of the present disclosure) and/or other molecules (e.g. target molecules, liposome-destabilising agents of the present disclosure).

As used herein, a ‘complex’ refers to a multi-molecular species formed by the association of two or more molecules through covalent or non-covalent interaction. In some embodiments, complexes according to the present disclosure are formed through non-covalent interaction, e.g. through electrostatic interaction (e.g. ionic bonding, hydrogen bonding) and/or Van der Waals forces.

Complexes comprising an aptamer and a target for the aptamer according to the present disclosure may be formed through non-covalent interaction between the aptamer and the target molecule.

Complexes comprising nucleic acids (e.g. comprising an aptamer and another nucleic acid) according to the present disclosure may be formed through hydrogen bonding between complementary base pairs. That is, the complex may be formed through base-pairing of one or more complementary sequence(s) of nucleotides on the molecules. The constituent molecules of such a complex may be referred to as having been ‘hybridised’.

In some aspects and embodiments, the present disclosure provides a complex comprising an aptamer specific for a target molecule as described herein, and an antisense nucleic acid to the aptamer specific for the liposome-destabilising agent described herein. The nucleic acid constituents of such a complex may form a complex as a consequence of hybridisation between a toehold nucleic acid sequence and a complementary sequence. In some embodiments, the toehold sequence is provided on the antisense nucleic acid to the aptamer specific for the liposome-destabilising agent, and has complementarity to a sequence of nucleotides of the aptamer specific for a target molecule.

In some aspects and embodiments, the present disclosure provides a complex comprising a liposome-destabilising agent described herein and an aptamer specific for the liposome-destabilising agent.

In some aspects and embodiments, the present disclosure provides a complex comprising a liposome-destabilising agent described herein and a composite aptamer, comprising an aptamer specific for the liposome-destabilising agent, and an aptamer specific for a target molecule. It will be appreciated that in such complexes, the complex is formed by interaction between the liposome-destabilising agent described herein and an aptamer specific for the liposome-destabilising agent.

Also provided by the present disclosure are complexes comprising the aptamer specific for a target molecule bound to the target molecule.

In some aspects and embodiments, the present disclosure provides a complex comprising: a composite aptamer comprising an aptamer specific for the liposome-destabilising agent, and an aptamer specific for a target molecule; and the target molecule. It will be appreciated that in such complexes, the complex is formed by interaction between the aptamer specific for target molecule and the target molecule.

Also provided by the present disclosure are complexes comprising the aptamer specific for a liposome-destabilising agent bound to the antisense nucleic acid to the aptamer specific for a liposome-destabilising agent.

In some aspects and embodiments, the present disclosure provides a complex comprising: a composite aptamer comprising an aptamer specific for the liposome-destabilising agent, and an aptamer specific for a target molecule; and an antisense nucleic acid to the aptamer specific for a liposome-destabilising agent. It will be appreciated that in such complexes, the complex is formed by interaction between the aptamer specific for the liposome-destabilising agent and the antisense nucleic acid to the aptamer specific for a liposome-destabilising agent.

### Liposome Encapsulating an Antisense Nucleic Acid to the Aptamer Specific for the Liposome-Destabilising Agent

Aspects and embodiments of the present disclosure relate to a liposome encapsulating an antisense nucleic acid to the aptamer specific for the liposome-destabilising agent, e.g. an antisense nucleic acid to the aptamer specific for the liposome-destabilising agent described herein.

The liposome encapsulating an antisense nucleic acid to the aptamer specific for the liposome-destabilising agent may also be referred to herein as an ‘amplification liposome’. In preferred embodiments, liberation of antisense nucleic acid to the aptamer specific for the liposome-destabilising agent from the amplification liposome results in the disruption of more complexes comprising the liposome-destabilising agent and the aptamer specific for the liposome-destabilising agent (i.e. through complementary base pairing between the aptamer specific for the liposome-destabilising agent and the antisense nucleic acid to the aptamer specific for the liposome-destabilising agent), liberating more liposome-destabilising agent, which in turn results in the destabilisation of further liposomes encapsulating a chemical entity for producing a detectable signal (through the destabilisation by the liposome-destabilising agent).

The liposome encapsulating an antisense nucleic acid to the aptamer specific for the liposome-destabilising agent is susceptible to inducible destabilisation. In the context of the liposome encapsulating an antisense nucleic acid to the aptamer specific for the liposome-destabilising agent, destabilisation of the liposome liberates the antisense nucleic acid to the aptamer specific for the liposome-destabilising agent.

The liposome encapsulating an antisense nucleic acid to the aptamer specific for the liposome-destabilising agent according to the present disclosure is preferably destabilised by release of the chemical entity for producing a detectable signal from the liposome encapsulating a chemical entity for producing a detectable signal. That is, the liposome preferably comprises a membrane susceptible to destabilisation as a consequence of release of the chemical entity for producing a detectable signal.

In some embodiments, destabilisation of the liposome encapsulating an antisense nucleic acid to the aptamer specific for the liposome-destabilising agent by the chemical entity for producing a detectable signal may be direct, e.g. resulting from interaction between the chemical entity and the liposomal membrane.

In some embodiments, destabilisation of the liposome encapsulating an antisense nucleic acid to the aptamer specific for the liposome-destabilising agent by the chemical entity for producing a detectable signal is indirect. In some embodiments, destabilisation occurs as a consequence of a chemical or physical change that occurs in a sample comprising the liposome following release of the chemical entity from the liposome encapsulating the chemical entity.

In preferred embodiments, the liposome encapsulating an antisense nucleic acid to the aptamer specific for the liposome-destabilising agent according to the present disclosure is susceptible to destabilisation as a consequence of an increase in the amount of non-encapsulated chemical entity for producing a detectable signal.

In any given embodiment of the articles and methods of the present disclosure, the composition of the membrane of the liposome encapsulating an antisense nucleic acid to the aptamer specific for the liposome-destabilising agent according to the present disclosure is selected so as to be susceptible to direct or indirect destabilisation by liberation of the chemical entity for producing a detectable signal from the liposome encapsulating a chemical entity for producing a detectable signal.

In some embodiments, the liposome encapsulating an antisense nucleic acid to the aptamer specific for the liposome-destabilising agent is destabilised by low pH, and the chemical entity for producing a detectable signal is or comprises an acidic species. In such embodiments, release of the chemical entity from the liposome encapsulating the chemical entity may lower the pH of the sample, which may in turn lead to destabilisation of the liposome encapsulating an antisense nucleic acid to the aptamer specific for the liposome-destabilising agent.

1. A computer-implemented method of determining the presence of a target species in a fluid sample, the computer-implemented method comprising:
receiving a captured image including an image capture portion of a sample vessel, the image capture portion exposing a fluid sample contained within the sample vessel, wherein an optical property of the fluid sample is indicative of the presence of the target species;
processing the captured image to generate optical property data representative of the optical property of the fluid sample; and
determining whether the target species is present in the fluid sample based on the optical property data.

2. The computer-implemented method according to claim 1, wherein:
the presence, expression or activity of the target species is associated with a status or progression of a disease; or
the upregulated expression or activity of the target species is associated with a status or progression of a disease.

3. The computer-implemented method according to claim 1, wherein:
the captured image includes an identification code, which encodes additional data about the test, the additional data including one or more of: an identity of the subject, a date/time at which the test is taken, a unique ID of the test session, information about the disease, information about the first reagent, information about the second reagent, and a test version;
the identification code is located in the image capture region; and
the captured image includes a fluid sample region in which the fluid sample is visible, and an identification code region in which the identification code is visible.

4. (canceled)

5. The computer-implemented method according to claim 3, wherein:
the image capture region of the sample vessel is defined by the identification code; and
the identification code includes a window through which the fluid sample is visible.

6. The computer-implemented method according to claim 1, wherein:
the identification code includes boundary features indicating the location of the edge of the identification code; and
processing the captured image comprises identifying the image capture region in the captured image by detecting the boundary features in the captured image.

7-8. (canceled)

9. The computer-implemented method according to claim 1, wherein:
the optical property is a colour of the fluid sample; and
processing the captured image comprises determining the colour of the fluid sample in the fluid sample region of the captured image.

10-11. (canceled)

12. The computer-implemented method according to claim 1, wherein:
determining whether the target species is present in the fluid sample comprises applying an analytical model to the optical property data, the output of the model being a value indicative of the presence and/or among of the target species in the fluid sample; and
either:
the input of the analytical model is the raw optical property data comprising data about the calibration features; or
the identification code includes one or more colour calibration features, the method further comprises a step of adjusting the optical property data based on the colour calibration features to generate adjusted optical property data, and the input of the analytical model is the adjusted optical property data.

13. (canceled)

14. A method of determining the presence of a target species in a fluid sample, the method including:
capturing an image including an image capture portion of a sample vessel, the image capture portion exposing a fluid sample contained within the sample vessel, wherein an optical property of the fluid sample is indicative of the presence of the target species; and
performing the computer-implemented method of claim 1.

15. The method according to claim 14, further comprising:
obtaining the fluid sample in the sample vessel, which comprises the steps of:
providing the sample vessel to a subject, the sample vessel containing at least a first reagent;
obtaining a sample of a bodily fluid from the subject in the sample vessel, the bodily fluid containing the target species to be tested;
mixing at least the first reagent with the bodily fluid in the sample vessel, to provide the fluid sample, wherein the optical property of the fluid sample depends on a degree of interaction between the first reagent and the target species.

16. The method according to claim 14, further comprising:
obtaining the fluid sample in the sample vessel, which comprises the steps of:
providing the sample vessel to a subject, the sample vessel containing at least a first reagent and a second reagent;
obtaining a sample of a bodily fluid from the subject in the sample vessel, the bodily fluid containing the target species to be tested;
mixing at least the first reagent with the bodily fluid in the sample vessel, wherein interaction between the first reagent and the target species generates an intermediate reagent; and
mixing the intermediate reagent with the second reagent, to generate the fluid sample wherein the optical property of the fluid sample depends on a degree of interaction between the intermediate reagent and the second reagent.

17. A sample vessel for collection of a bodily fluid from a subject, and for preparation of a fluid sample, the sample vessel comprising:
a sample collection assembly, including a sample collection element which is configured to receive and retain a sample of a bodily fluid from a subject; and
a containment component including: an outer housing; an inner mechanism located inside the outer housing, the inner mechanism having an inner surface defining a cavity arranged to receive the sample collection element of the sample collection assembly; and a first reagent chamber;
wherein the sample collection assembly is positionable in the cavity in a testing position in which the sample collection element is in fluid communication with the first reagent chamber.

18. The sample vessel according to claim 17, wherein:
the sample collection element comprises an absorbent body, and when the sample collection element is in the testing position, the absorbent body is in a compressed state in which it is configured to release the absorbed bodily fluid into the first reagent chamber.

19. The sample vessel according to claim 17, wherein:
the containment component comprises a locking mechanism configured to limit or prevent relative axial movement between the sample collection assembly and the inner mechanism when the sample collection assembly is in the testing position.

20. The sample vessel according to claim 17, wherein:
in a first relative configuration of the inner mechanism and the outer housing of the containment component, the first reagent chamber is defined by a first chamber-defining surface of the outer housing and a first chamber-defining surface of the inner mechanism.

21. The sample vessel according to claim 14, wherein:
in the first relative configuration of the inner mechanism and the outer housing of the containment component, the containment chamber further comprises a second reagent chamber.

22. The sample vessel according to claim 15, wherein:
the inner mechanism is movable relative to the outer housing, between the first relative configuration and a second relative configuration in which the inner mechanism and the outer housing are in the second relative configuration, the first reagent chamber and the second reagent chamber are in fluid communication with each other.

23. The sample vessel according to claim 16, wherein:
the inner mechanism is movable relative to the outer housing from the first relative configuration to the second relative configuration only under the application of a force of sufficient magnitude.

24. (canceled)

25. The sample vessel according to claim 16, wherein:
the containment component further comprises a second locking mechanism, configured to prevent relative axial movement between the inner mechanism and the outer housing, when they are in the second relative configuration.

26. The sample vessel according to claim 15, wherein:
the first reagent chamber contains a first reagent; and
the second reagent chamber contains a second reagent.

27. The sample vessel according to claim 17, wherein:
the outer housing includes a peripheral wall including a transparent or translucent image capture portion; and
the image capture region further comprises an identification code, wherein an outer boundary of the image capture region is defined by the identification code and the identification code includes a window through which the transparent or translucent portion of the peripheral wall is visible.

28-31. (canceled)
